(19) United States
(12) Patent Application Publication
(10) Pub. No.: US 2007/0087961 A1
(43) Pub. Date: Apr. 19, 2007

(54) Conjugates of hydroxyalkyl starch and erythropoietin

(76) Inventors: Wolfram Eichner, Butzbach (DE); Katharina Lutterbeck, Friedberg (DE); Norbert Zander, Meine (DE); Ronald Frank, Meine Grassel (DE); Harald Conradt, Braunshweig (DE); Helmut Knoller, Friedberg (DE)

Correspondence Address: FISH & RICHARDSON P.C., PO BOX 1022, MINNEAPOLIS, MN, 55440-1022, US

(21) Appl. No.: 11/530,326

(22) Filed: Sep. 8, 2006

Related U.S. Application Data
(63) Continuation-in-part of application No. PCT/EP05/02639, filed on Mar. 11, 2005.
(60) Provisional application No. 60/552,119, filed on Mar. 11, 2004.

(51) Int. Cl. A61K 38/18; C07K 14/505

(52) U.S. Cl. 514/8; 530/395

(57) ABSTRACT

Conjugates of hydroxyethyl starch and erythropoietin are provided. The conjugates comprise a linking compound that is covalently linked to erythropoietin and covalently linked to hydroxyethyl starch. Methods of producing the conjugates, and their use, also are provided.

### CROSS-REFERENCE TO RELATED APPLICATIONS

This application is a continuation-in-part and claims benefit under 35 U.S.C. § 119(a) of International Application No. PCT/EP2005/002639 having an International Filing Date of Mar. 11, 2005, which published in English as International Publication Number WO 2005/092369, and which claims the benefit of priority of U.S. Provisional Application Ser. No. 60/552,119, filed on Mar. 11, 2004.

### TECHNICAL FIELD

The present invention relates to conjugates of erythropoietin (EPO) and hydroxyalkyl starch (HAS), in particular hydroxyethyl starch (HES). These conjugates comprise a linking compound which is covalently linked to EPO and covalently linked to HAS. The present invention also relates to the method of producing these conjugates and their use.

### BACKGROUND

EPO is a glycoprotein hormone necessary for the maturation of erythroid progenitor cells into erythrocytes. In human adults, it is produced in the kidney. EPO is essential in regulating the level of red blood cells in the circulation. Conditions marked by low levels of tissue oxygen provoke an increased biosynthesis of EPO, which in turn stimulates erythropoiesis. A loss of kidney function as it is seen in chronic renal failure, for example, typically results in decreased biosynthesis of EPO and a concomitant reduction in red blood cells.

Erythropoietin is an acid glycoprotein hormone of approximately 34,000 D. Human erythropoietin is a 166 amino acid polypeptide that exists naturally as a monomer (Lin et al., 1985, PNAS 82, 7580-7584, EP 148 605 B2, EP 411 678 B2). The identification, cloning and expression of genes encoding erythropoietin are described, e.g., in U.S. Pat. No. 4,703,008. The purification of recombinant erythropoietin from cell culture medium that supported the growth of mammalian cells containing recombinant erythropoietin plasmids, for example, is described in U.S. Pat. No. 4,667,016.

It is generally believed in this technical field that the biological activity of EPO in vivo mainly depends on the degree of sialic acids bound to EPO (see e.g. EP 428 267 B1). Theoretically, 14 molecules of sialic acid can be bound to one molecule EPO at the terminal ends of the carbohydrate side chains linked to N- and O-glycosylation sites. Highly sophisticated purification steps are necessary to obtain highly sialylated EPO preparations.

For further detailed information on erythropoietin see Krantz, Erythropoietin, 1991, Blood, 77(3):419-34 (Review) and Cerami, Beyond erythropoiesis: novel applications for recombinant human erythropoietin, 2001, Semin Hematol., (3 Suppl 7):33-9 (Review).

A well-known problem with the application of polypeptides and enzymes is that these proteins often exhibit an unsatisfactory stability. Especially, erythropoietin has a relatively short plasma half live (Spivak and Hogans, 1989, Blood 73, 90; McMahon et al., 1990, Blood 76, 1718). This means that therapeutic plasma levels are rapidly lost and repeated intravenous administrations must be carried out. Furthermore, in certain circumstances an immune response against the peptides is observed.

It is generally accepted that the stability of polypeptides can be improved and the immune response against these polypeptides is reduced when the polypeptides are coupled to polymeric molecules. WO 94/28024 discloses that physiologically active polypeptides modified with polyethyleneglycol (PEG) exhibit reduced immunogenicity and antigenicity and circulate in the bloodstream considerably longer than unconjugated proteins, i.e. have a reduced clearance rate.

However, PEG-drug conjugates exhibit several disadvantages, e.g. they do not exhibit a natural structure which can be recognized by elements of in vivo degradation pathways. Therefore, apart from PEG-conjugates, other conjugates and protein polymers have been produced. A plurality of methods for the cross-linking of different proteins and macromolecules such as polymerase have been described in the literature (see e.g. Wong, Chemistry of protein conjugation and cross-linking, 1993, CRCS, Inc.).

WO 03/074087 relates to a method of coupling proteins to a starch-derived modified polysaccharide. The binding action between the protein and the polysaccharide, hydroxyalkyl starch, is a covalent linkage which is formed between the terminal aldehyde group or a functional group resulting from chemical modification of said terminal aldehyde group of the hydroxy alkyl starch molecule, and a functional group of the protein. As reactive group of the protein, amino groups, thio groups and carboxyl groups are disclosed. Specifically, WO 03/074087 is silent on the possibility of coupling hydroxy alkyl starch to a carbohydrate moiety of the protein. Moreover, whilst a vast variety of possibilities of different linkages is given in the form of many lists, including different functional groups, theoretically suitable different linker molecules, and different chemical procedures, the working examples describe only two alternatives: first, an oxidized hydroxyethyl starch is used and coupled directly to proteins using ethyldimethylaminopropyl carbodiimide (EDC) activation, or a non-oxidized hydroxyethyl starch is used and coupled directly, i.e. without linking compound to a protein forming a Schiff's base which is subsequently reduced to the respective amine. Thus, the working examples of WO 03/074087 do not disclose a single conjugate comprising hydroxyethyl starch, the protein, and one or more linker molecules. Additionally, WO 03/074087 does not contain any information as to a linking compound comprising an aminooxy, i.e. a hydroxylamino group.

### SUMMARY

Consequently, it is an object of the present invention to provide polypeptide derivatives, especially erythropoietin derivatives, having a high biological activity in vivo which can be easily produced and at reduced costs.

Therefore, it is another object of the present invention to provide conjugates of erythropoietin and hydroxyethyl starch with improved specific in vivo activity.

It is yet another object of the present invention to provide a method for preparing these conjugates of erythropoietin and hydroxyethyl starch with improved specific in vivo activity.

It is still another object of the present invention to provide a method for improving the specific in vivo activity of conjugates of erythropoietin and hydroxyethyl starch by changing the characteristics of the hydroxyethyl starch used for the production of the conjugates.

Therefore, the present invention relates to a conjugate of erythropoietin and hydroxyethyl starch, comprising a homobifunctional crosslinking compound having two hydroxylamino groups, one of which is covalently linked to a carbohydrate moiety of the erythropoietin and one of which is covalently linked to the hydroxyethyl starch, wherein the hydroxyethyl starch has a mean molecular weight of at least 40 kD and a degree of substitution of at least 0.6.

Furthermore, the present invention also relates to a method of producing a conjugate of erythropoietin and hydroxyethyl starch, said method comprising reacting the hydroxyethyl starch with a homobifunctional crosslinking compound having two hydroxylamino groups to give a hydroxylamino functionalized hydroxyethyl starch derivative, and reacting the hydroxylamino group of said derivative with a carbohydrate moiety of the erythropoietin, wherein the hydroxyethyl starch has a mean molecular weight of at least 40 kD and a degree of substitution of at least 0.6.

Furthermore, the present invention also relates to a conjugate obtainable by the method according to the invention.

In one aspect, this document features a method of producing a conjugate of erythropoietin and hydroxyethyl starch. The method can include reacting the hydroxyethyl starch with a crosslinking compound having two hydroxylamino groups to give a hydroxylamino functionalized hydroxyethyl starch derivative, and reacting the hydroxylamino group of the derivative with a carbohydrate moiety of the erythropoietin, wherein the hydroxyethyl starch has a mean molecular weight of at least 40 kD and a degree of substitution of at least 0.6. The hydroxyethyl starch can have a mean molecular weight of at least 50 kD and a degree of substitution of at least 0.7 (e.g., a degree of substitution of about 0.7 to 0.8). The crosslinking compound can be O-[2-(2-aminooxy-ethoxy)-ethyl]-hydroxylamine.

The hydroxyethyl starch can be reacted with the crosslinking compound in an aqueous medium. The reaction can be carried out at a pH of from 4.5 to 6.5. The reaction can be carried out at a temperature of from 20 to 25° C.

The hydroxylamino functionalized hydroxyethyl starch derivative can be reacted with the carbohydrate moiety of the erythropoietin in an aqueous medium. The reaction can be carried out at a pH of from 4.5 to 6.5. The reaction can be carried out at a temperature of from 20 to 25° C.

The carbohydrate moiety can be an oxidized terminal saccharide unit of a carbohydrate side chain of the erythropoietin, preferably a terminal sialic acid. The terminal saccharide unit can be oxidized, optionally after partial or complete enzymatic and/or chemical removal of the terminal sialic acid. The terminal saccharide unit can be galactose.

The carbohydrate moiety can be comprised in a carbohydrate side chain of erythropoietin which was attached to the erythropoietin via N- and/or O-linked glycosylation during its production in mammalian cells (e.g., human cells), insect cells, or yeast cells.

In another aspect, this document features a conjugate of erythropoietin and hydroxyethyl starch, obtainable by a method as described herein.

In another aspect, this document features a conjugate comprising hydroxyethyl starch, a crosslinking compound and erythropoietin, wherein the crosslinking compound is linked via an oxime linkage and/or an oxyamino group to the hydroxyethyl starch and via an oxime linkage to the carbohydrate moiety of the erythropoietin, and wherein the hydroxyethyl starch has a mean molecular weight of at least 40 kD and a degree of substitution of at least 0.6. The hydroxyethyl starch can have a mean molecular weight of at least 50 kD and a degree of substitution of at least 0.7 (e.g., a degree of substitution of about 0.7 to about 0.8). The conjugate can comprise a crosslinking compound having had two hydroxylamino groups, one of which being covalently linked to a carbohydrate moiety of the erythropoietin and one of which being covalently linked to the hydroxyethyl starch. The crosslinking compound can be O-[2-(2-aminooxy-ethoxy)-ethyl]-hydroxylamine. The erythropoietin can have the amino acid sequence of human erythropoietin. The carbohydrate moiety can be comprised in a carbohydrate side chain attached to the erythropoietin via N- and/or O-linked glycosylation, the erythropoietin comprising at least one carbohydrate side chain. The at least one carbohydrate side chain can have been attached to the erythropoietin during production of the erythropoietin in mammalian cells (e.g., human cells), insect cells, yeast cells, transgenic animals or transgenic plants. The carbohydrate moiety can be an oxidized galactose residue or a sialic acid residue.

In yet another aspect, this document features a method for screening for a conjugate of erythropoietin and hydroxyalkyl starch, preferably hydroxyethyl starch, having improved in vivo activity compared to native erythropoietin, the method comprising the steps of (i) providing a candidate conjugate; (ii) testing the in vivo activity in comparison with native erythropoietin, wherein the mean molecular weight MW is varied in the range of from 1 to 300 kD and the degree of substitution DS is varied in the range of from 0.1 to 1.0, and wherein these parameters are simultaneously increased compared to a given combination of parameters. The given combination of parameters can be a mean molecular weight MW of about 10 kD and a degree of substitution DS of about 0.4. The method can further include the step of incorporating the candidate conjugate into a therapeutic or prophylactic composition.

In still another aspect, this document features a method for the treatment of a human or animal body, comprising administering a conjugate as described herein to a human or animal in need of treatment.

This document also features a pharmaceutical composition comprising in a therapeutically effective amount a conjugate as described herein. The pharmaceutical composition can further contain at least one pharmaceutically acceptable diluent, adjuvant, and/or carrier.

In another aspect, this document features a composition for the treatment of anemic disorders or hematopoietic dysfunction disorders or diseases related thereto, comprising a conjugate as described herein.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows a protein gel comparing Q-Sepharose eluates (HES-modified and unmodified EPO) before and after digestion with polypeptide N-glycosidase. In each case, an aliquot corresponding to 20 μg EPO protein was applied onto the gel. The protein band at kD 21 represents the O-glycosylated EPO species without HES-modification, the diffuse band migration above represents EPO forms with HES modification at the O-glycosylation site at Ser126.

FIG. 2 shows a protein gel comparing de-N-glycosylated EPO proteins after RP-HPLC before (−) and after (+) mild acid hydrolysis by SDS-PAGE analysis. In each case, 1.5% of the different eluates were treated with 5 mM H2SO4 at 83° C. for 90 min or were left untreated and were applied after ethanol precipitation applied onto the gel in sample buffer.

FIG. 3 shows an SDS page analysis of HES-EPO conjugates, produced according to Example 3.2. For gel electrophoresis, a XCell Sure Lock Mini Cell (Invitrogen GmbH, Karlsruhe, D) and a Consort E143 power supply (CONSORTnv, Turnhout, B) were employed. A 10% Bis-Tris gel together with a MOPS SDS running buffer at reducing conditions (both Invitrogen GmbH, Karlsruhe, D) were used according to the manufacturer's instruction.

Lane A: Protein marker SeeBlue® Plus2 (Invitrogen GmbH, Karlsruhe, D) Molecular weight marker from top to bottom: 188 kD, 98 kD, 62 kD, 49 kD, 38 kD, 28 kD, 17 kD, 14 kD, 6 kD, 3 kD

| | |
|---|---|
| Lane B: | Crude reaction product Example 3.2(a) |
| Lane C: | Crude reaction product Example 3.2(b) |
| Lane E: | Crude reaction product Example 3.2(d) |
| Lane F: | Crude reaction product Example 3.2(c) |
| Lane G: | Oxidized EPO according to Example 2. |

FIG. 4 is a graph plotting HPAEC-PAD analysis of native N-linked oligosaccharides from untreated, periodate oxidised and HES-modified EPO. 1 represents total oligosaccharides from the EPO starting material (A14), 2 represents oligosaccharides obtained from the Q-sepharose purified product modified with HES 10/0.4 (A20), 3 represents oligosaccharides obtained from the Q-sepharose purified product modified with HES 10/0.7 (A21), 5 represents oligosaccharides obtained from the Q-sepharose purified product modified with HES 50/0.7 (A23), 6 represents oligosaccharides obtained from the Q-sepharose purified periodate treated EPO (A24), and 7 represents oligosaccharides obtained from the Q-sepharose purified product modified with HES 50/0.4 (A25). 0 represents the neutral oligosaccharide fraction, I represents the mono-charged oligosaccharide fraction (1 sialic acid), II represents the di-charged oligosaccharide fraction (2 sialic acid), III represents the tri-charged oligosaccharide fraction (3 sialic acid), and IV represents the tetra-charged oligosaccharide fraction (4 sialic acid).

FIG. 5 is a graph plotting HPAEC-PAD analysis of N-linked oligosaccharides after mild acid treatment from untreated, periodate oxidised and HES-modified EPO (see Example 6.3(b)). 1 represents N-acetylneurarminic acid, 2 represents a diantennary structure+alpha 1-6 linked fucose, 3 represents a triantennary structure+alpha 1-6 linked fucose, 4 represents a triantennary structure (isomer), +alpha 1-6 linked fucose, 5 represents a tetraantennary structure, +alpha 1-6 linked fucose, 6 represents a tetraantennary structure plus 1 N-acetyllactosamin repeat+alpha 1-6 linked fucose, and 7 represents a tetraantennary structure plus 2 N-acetyllactosamin repeat+alpha 1-6 linked fucose. 0 represents oligosaccharides after mild acid hydrolysis from EPO starting material (A14), 1 represents oligosaccharides after mild acid hydrolysis from EPOmodified with HES 10/0.4 (A20), II represents oligosaccharides after mild acid hydrolysis from EPO modified with HES 10/0.7 (A21), IV represents oligosaccharides after mild acid hydrolysis from EPO modified with HES 50/0.7 (A23), V represents oligosaccharides after mild acid hydrolysis from EPO modified by mild periodate oxidation (A24), and VI represents oligosaccharides after mild acid hydrolysis from EPO modified with HES 50/0.4 (A25).

### DETAILED DESCRIPTION

In the context of the present invention, the term “hydroxyethyl starch” (HES) refers to a starch derivative which has been substituted by at least one hydroxyethyl group. A preferred hydroxyethyl starch of the present invention has a structure according to formula (I) 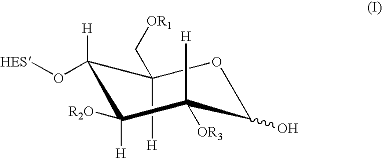
wherein the reducing end of the starch molecule is shown in the non-oxidized form and the terminal saccharide unit is shown in the acetal form which, depending on e.g. the solvent, may be in equilibrium with the aldehyde form. In formula (I), the saccharide ring described explicitly and the residue denoted as HES′ together represent the preferred hydroxyethyl starch molecule. The other saccharide ring structures comprised in HES′ may be the same as or different from the explicitly described saccharide ring.

The term “hydroxyethyl starch” as used in the present invention is not limited to compounds where the terminal carbohydrate moiety comprises hydroxyethyl groups R₁, R₂, and/or R₃ as depicted, for the sake of brevity, in formula (I), but also refers to compounds in which at least one hydroxy group present anywhere, either in the terminal carbohydrate moiety and/or in the remaining part of the starch molecule, HES′, is substituted by a hydroxyethyl group R₁, R₂, or R₃.

The at least one hydroxyethyl group comprised in HES may contain two or more hydroxy groups. According to a preferred embodiment, the at least one hydroxyethyl group comprised in HES contains one hydroxy group.

The expression “hydroxyethyl starch” also includes derivatives wherein the ethyl group is mono- or polysubstituted. In this context, it is preferred that the ethyl group is substituted with a halogen, especially fluorine, or with an aryl group. Furthermore, the hydroxy group of a hydroxyethyl group may be esterified or etherified.

Techniques for the esterification of starch are known in the art (see e.g. Klemm D. et al, Comprehensive Cellulose Chemistry Vol. 2, 1998, Whiley-VCH, Weinheim, N.Y., especially chapter 4.4, Esterification of Cellulose (ISBN 3-527-29489-9).

As far as the residues R₁, R₂ and R₃ according to formula (I) are concerned, there are no specific limitations. According to a preferred embodiment, R₁, R₂ and R₃ are independently hydrogen or a 2-hydroxyethyl group.

Hydroxyethyl starch (HES) is a derivative of naturally occurring amylopectin and is degraded by alpha-amylase in the body. HES is a substituted derivative of the carbohydrate polymer amylopectin, which is present in corn starch at a concentration of up to 95% by weight. HES exhibits advantageous biological properties and is used as a blood volume replacement agent and in hemodilution therapy in the clinics (Sommermeyer et al., 1987, Krankenhauspharmazie, 8(8), 271-278; and Weidler et al., 1991, Arzneim-Forschung/Drug Res., 41, 494-498).

Amylopectin consists of glucose moieties, wherein in the main chain alpha-1,4-glycosidic bonds are present and at the branching sites alpha-1,6-glycosidic bonds are found. The physical-chemical properties of this molecule are mainly determined by the type of glycosidic bonds. Due to the nicked alpha-1,4-glycosidic bond, helical structures with about six glucose-monomers per turn are produced. The physico-chemical as well as the biochemical properties of the polymer can be modified via substitution. The introduction of a hydroxyethyl group can be achieved via alkaline hydroxyethylation. By adapting the reaction conditions it is possible to exploit the different reactivity of the respective hydroxy group in the unsubstituted glucose monomer with respect to a hydroxyethylation. Owing to this fact, the skilled person is able to influence the substitution pattern to a limited extent.

HES is mainly characterized by the molecular weight distribution and the degree of substitution. There are two possibilities of describing the substitution degree:
1. The degree of substitution can be described relatively to the portion of substituted glucose monomers with respect to all glucose moieties.
2. The degree of substitution can be described as the molar substitution, wherein the number of hydroxyethyl groups per glucose moiety are described.

In the context of the present invention, the degree of substitution, denoted as DS, relates to the molar substitution, as described above (see also Sommermeyer et al., 1987, Krankenhauspharmazie, 8(8), 271-278, as cited above, in particular p. 273).

HES preparations are present as polydisperse compositions, wherein each molecule differs from the other with respect to the polymerisation degree, the number and pattern of branching sites, and the substitution pattern. HES is therefore a mixture of compounds with different molecular weight. Consequently, a particular HES solution is determined by average molecular weight with the help of statistical means. In this context, Mₙ, is calculated as the arithmetic mean depending on the number of molecules. Alternatively, M_{w} (or M_{W}), the weight mean, represents a unit which depends on the mass of the HES.

In the context of the present invention, the hydroxyethyl starch has a mean molecular weight (weight mean) of at least about 40 kD and degree of substitution DS of at least about 0.6.

According to more preferred embodiments of the present invention, hydroxyethyl starch has a mean molecular weight (weight mean) of at least about 40 kD and degree of substitution DS of greater than about 0.6 or a mean molecular weight (weight mean) of greater than about 40 kD and degree of substitution DS of at least about 0.6. According to an even more preferred embodiment, hydroxyethyl starch has a mean molecular weight (weight mean) of greater than about 40 kD and degree of substitution DS of greater than about 0.6.

According to a still further preferred embodiment of the present invention, the hydroxyethyl starch has a mean molecular weight (weight mean) of at least about 50 or 100 kD and degree of substitution DS of at least about 0.7, more preferably having a mean molecular weight (weight mean) of about 50 or 100 kD and degree of substitution DS of at least about 0.7 or having a mean molecular weight (weight mean) of at least about 50 or 100 kD and degree of substitution DS of about 0.7 and even more preferably having a mean molecular weight (weight mean) of about 50 or 100 kD and degree of substitution DS of about 0.7.

Therefore, the present invention also relates to a conjugate and a method as described above, wherein the hydroxyethyl starch has a mean molecular weight of at least 50 kD and a degree of substitution of at least 0.7.

Accordingly, the present invention also relates to a conjugate and a method as described above, wherein the hydroxyethyl starch has a mean molecular weight of about 50 kD and a degree of substitution of about 0.7.

According to another embodiment of the present invention, the hydroxyethyl starch has a mean molecular weight (weight mean) of at least about 50 kD and degree of substitution DS of at least about 0.8, more preferably having a mean molecular weight (weight mean) of about 50 kD and degree of substitution DS of at least about 0.8 or having a mean molecular weight (weight mean) of at least about 50 kD and degree of substitution DS of about 0.8 and even more preferably having a mean molecular weight (weight mean) of about 50 kD and degree of substitution DS of about 0.8.

Further described is a hydroxyethyl starch having a mean molecular weight (weight mean) of at least about 120 kD and degree of substitution DS of at least about 0.6, such as a mean molecular weight of about 120 kD and degree of substitution DS of at least about 0.6, or a mean molecular weight of about 120 kD and degree of substitution DS of at least about 0.7, or a mean molecular weight of about 120 kD and degree of substitution DS of at least about 0.8, or a mean molecular weight of about 120 kD and degree of substitution DS of at least about 0.9, or a mean molecular weight of about 130 kD and degree of substitution DS of at least about 0.6, or a mean molecular weight of about 130 kD and degree of substitution DS of at least about 0.7, or a mean molecular weight of about 130 kD and degree of substitution DS of at least about 0.8, or a mean molecular weight of about 130 kD and degree of substitution DS of at least about 0.9, or a mean molecular weight of about 140 kD and degree of substitution DS of at least about 0.6, or a mean molecular weight of about 140 kD and degree of substitution DS of at least about 0.7, or a mean molecular weight of about 140 kD and degree of substitution DS of at least about 0.8, or a mean molecular weight of about 140 kD and degree of substitution DS of at least about 0.9.

Therefore, the present invention also relates to a conjugate and a method as described above, wherein the hydroxyethyl starch has a mean molecular weight of about 130 kD and a degree of substitution of about 0.6 or of about 0.7 or of about 0.8 or of about 0.9.

The term “about 40 kD” as used in the context of the present relates to a mean molecular weight in the range of from 38 kD to 42 kD, more preferably in the range of from 39 kD to 41 kD.

The term “about 50 kD” as used in the context of the present relates to a mean molecular weight in the range of from 48 kD to 52 kD, more preferably in the range of from 49 kD to 51 kD.

The term “about 120 kD” as used in the context of the present relates to a mean molecular weight in the range of from 116 kD to 124 kD, more preferably in the range of from 118 kD to 122 kD.

The term “about 130 kD” as used in the context of the present relates to a mean molecular weight in the range of from 126 kD to 134 kD, more preferably in the range of from 128 kD to 132 kD.

The term “about 140 kD” as used in the context of the present relates to a mean molecular weight in the range of from 136 kD to 144 kD, more preferably in the range of from 138 kD to 142 kD.

The term “about 0.6” as used in the context of the present with regard to DS relates to a degree of substitution in the range of greater than 0.55 to 0.65, more preferably in the range of from 0.58 to 0.62.

The term “about 0.7” as used in the context of the present with regard to DS relates to a degree of substitution in the range of greater than 0.65 to 0.75, more preferably in the range of from 0.68 to 0.72.

The term “about 0.8” as used in the context of the present with regard to DS relates to a degree of substitution in the range of greater than 0.75 to 0.85, more preferably in the range of from 0.78 to 0.82.

The term “about 0.9” as used in the context of the present with regard to DS relates to a degree of substitution in the range of greater than 0.85 to 0.95, more preferably in the range of from 0.88 to 0.92.

As far as the ratio of C₂: C₆ substitution of the hydroxyethyl starch is concerned, said substitution is preferably in the range of from 2 to 20, more preferably in the range of from 2 to 15 and even more preferably in the range of from 3 to 12.

The term “mean molecular weight” as used in the context of the present invention relates to the weight as determined according the LALLS-(low angle laser light scattering)-GPC method as described in Sommermeyer et al., 1987, Krankenhauspharmazie, 8(8), 271-278; and Weidler et al., 1991, Arzneim-Forschung/Drug Res., 41, 494-498. For mean molecular weights of 10 kD and smaller, additionally, the calibration was carried out classically with a standard which had been previously qualified by LALLS-GPC.

The EPO used according to the present invention can be of any human (see e.g. Inoue, Wada, Takeuchi, 1994, An improved method for the purification of human erythropoietin with high in vivo activity from the urine of anemic patients, Biol Pharm Bull. 17(2), 1804; Miyake, Kung, Goldwasser, 1977, Purification of human erythropoietin., J Biol. Chem., 252(15), 5558-64) or another mammalian source and can be obtained by purification from naturally occurring sources like human kidney, embryonic human liver or animal, preferably monkey kidney. Furthermore, the expression “erythropoietin” or “EPO” encompasses also an EPO variant wherein one or more amino acids (e.g. 1 to 25, preferably 1 to 10, more preferred 1 to 5, most preferred 1 or 2) have been exchanged by another amino acid and which exhibits erythropoietic activity (see e.g. EP 640 619 B1). The measurement of erythropoietic activity is described in the art (for measurement of activity in vitro see e.g. Fibi et al., 1991, Blood, 77, 1203 ff; Kitamura et al, 1989, J. Cell Phys., 140, 323-334; for measurement of EPO activity in vivo see Ph. Eur. 2001, 911-917; Ph. Eur. 2000, 1316 Erythropoietini solutio concentrata, 780-785; European Pharmacopoeia (1996/2000); European Pharmacopoeia, 1996, Erythropoietin concentrated solution, Pharmacuropa., 8, 371-377; Fibi, Hermentin, Pauly, Lauffer, Zettlmeissl., 1995, N- and O-glycosylation muteins of recombinant human erythropoietin secreted from BHK-21 cells, Blood, 85(5), 1229-36; (EPO and modified EPO forms were injected into female NMRI mice (equal amounts of protein 50 ng/mouse) at day 1, 2 and 3 blood samples were taken at day 4 and reticulocytes were determined)). Further publications where tests for the measurement of the activity of EPO are Barbone, Aparicio, Anderson, Natarajan, Ritchie, 1994, Reticulocytes measurements as a bioassay for erythropoictin, J. Pharm. Biomed. Anal., 12(4), 515-22; Bowen, Culligan, Beguin, Kendall, Villis, 1994, Estimation of effective and total erythropoiesis in myelodysplasia using serum transferrin receptor and erythropoietin concentrations, with automated reticulocyte parameters, Leukemi, 8(1), 151-5; Delorme, Lorenzini, Giffin, Martin, Jacobsen, Boone, Elliott, 1992, Role of glycosylation on the secretion and biological activity of erythropoietin, Biochemistry, 31(41), 9871-6; Higuchi, Oh-eda, Kuboniwa, Tomonoh, Shimonaka, Ochi, 1992; Role of sugar chains in the expression of the biological activity of human erythropoietin, J. Biol. Chem., 267(11), 7703-9; Yamaguchi, Akai, Kawanishi, Ueda, Masuda, Sasaki, 1991, Effects of site-directed removal of N-glycosylation sites in human erythropoietin on its production and biological properties, J. Biol. Chem., 266(30), 20434-9; Takeuchi, Inoue, Strickland, Kubota, Wada, Shimizu, Hoshi, Kozutsumi, Takasaki, Kobata, 1989, Relationship between sugar chain structure and biological activity of recombinant human erythropoietin produced in Chinese hamster ovary cells, Proc. Natl. Acad. Sci. USA, 85(20), 7819-22; Kurtz, Eckardt, 1989, Assay methods for erythropoietin, Nephron., 51(1), 11-4 (German); Zucali, Sulkowski, 1985, Purification of human urinary erythropoietin on controlled-pore glass and silicic acid, Exp. Hematol., 13(3), 833-7; Krystal, 1983, Physical and biological characterization of erythroblast enhancing factor (EEF), a late acting erythropoetic stimulator in serum distinct from erythropoietin, Exp. Hematol., 11(1), 18-31.

According to a preferred embodiment of the present invention, EPO is recombinantly produced. This includes the production in eukaryotic or prokaryotic cells, preferably mammalian, insect, yeast, bacterial cells or in any other cell type which is convenient for the recombinant production of EPO. Furthermore, the EPO may be expressed in transgenic animals (e.g. in body fluids like milk, blood, etc.), in eggs of transgenic birds, especially poultry, preferred chicken, or in transgenic plants.

Therefore, the present invention also relates to a conjugate and a method as described above, wherein EPO is recombinantly produced.

The recombinant production of a polypeptide is known in the art. In general, this includes the transfection of host cells with an appropriate expression vector, the cultivation of the host cells under conditions which enable the production of the polypepfide and the purification of the polypeptide from the host cells. For detained information see e.g. Krystal, Panlkratz, Farber, Smart, 1986, Purification of human erythropoietin to homogeneity by a rapid five-step procedure, Blood, 67(1), 71-9; Quelle, Caslake, Burkert, Wojchowski, 1989, High-level expression and purification of a recombinant human erythropoietin produced using a baculovirus vector, Blood, 74(2), 652-7; EP 640 619 B1 and EP 668 351 B1.

In a preferred embodiment, the EPO has the amino acid sequence of human EPO (see EP 148 605 B2). Therefore, the present invention also relates to a conjugate and a method as described above, wherein EPO has the amino acid sequence of human EPO.

The EPO may comprise one or more carbohydrate side chains (preferably 1-4, preferably 4) attached to the EPO via N- and/or O-linked glycosylation, i.e. the EPO is glycosylated. Usually, when EPO is produced in eukaryotic cells, the polypeptide is posttranslationally glycosylated. Consequently, the carbohydrate side chains may have been attached to the EPO during biosynthesis in mammalian, especially human, insect or yeast cells. The structure and properties of glycosylated EPO have been extensively studied in the art (see EP 428 267 B1; EP 640 619 B1; Rush, Derby, Smith, Merry, Rogers, Rohde, Katta, 1995, Microheterogeneity of erythropoietin carbohydrate structure, Anal Chem., 67(8), 1442-52; Takeuchi, Kobata, 1991, Structures and functional roles of the sugar chains of human erythropoietins, Glycobiology, 1(4), 337-46 (Review).

Therefore, the present invention also relates to a conjugate and a method as described above, wherein the carbohydrate moiety is comprised in a carbohydrate side chain which was attached to the erythropoietin via N- and/or O-linked glycosylation, the erythropoietin comprising at least one carbohydrate side chain.

Thus, the present invention also relates to a conjugate and a method as described above, wherein the at least one carbohydrate side chain was attached to the erythropoietin during the production of the erythropoietin in mammalian, especially human cells, insect cells, yeast cells, transgenic animals or transgenic plants

According to the present invention, a hydroxylamino group of the crosslinking compound is linked to a carbohydrate moiety of the erythropoietin. In the context of the present invention, the term “carbohydrate moiety” refers to hydroxyaldehydes or hydroxyketones as well as to chemical modifications thereof (see Römpp Chemielexikon, Thieme Verlag Stuttgart, Germany, 9^{th} edition 1990, Volume 9, pages 2281-2285 and the literature cited therein). Furthermore, it also refers to derivatives of naturally occuring carbohydrate moieties like glucose, galactose, mannose, sialic acid and the like. The term also includes chemically oxidized naturally occuring carbohydrate moieties wherein the ring structure has been opened.

Thus, in the context of the present invention, the hydroxylamino group is linked to an aldehyde group or a keto group of the carbohydrate moiety, especially preferably to an aldehyde group of the carbohydrate moiety.

Therefore, the present invention also relates to a method and a conjugate as described above, wherein the crosslinking compound is linked via an oxime linkage to the hydroxyethyl starch and to the carbohydrate moiety of the erythropoietin.

The carbohydrate moiety may be linked directly to the EPO polypeptide backbone. Preferably, the carbohydrate moiety is part of a carbohydrate side chain. In this case, further carbohydrate moieties may be present between the carbohydrate moiety to which the hydroxylamino group is linked and the EPO polypeptide backbone. More preferably, the carbohydrate moiety is the terminal moiety of a carbohydrate side chain.

In a more preferred embodiment, the hydroxylamino group is linked to a galactose residue of a carbohydrate side chain, preferably the terminal galactose residue of a carbohydrate side chain. This galactose residue can be made available for conjugation by removal of terminal sialic acids, followed by oxidation.

In a still further preferred embodiment, the hydroxylamino group is linked to a preferably oxidized sialic acid residue of a carbohydrate side chains, preferably the terminal sialic acid residue of a carbohydrate side chain.

The EPO may comprise one or more carbohydrate side chains attached to the EPO via N- and/or O-linked glycosylation, i.e. the EPO is glycosylated. Unusually, when EPO is produced in eukaryotic cells, the polypeptide is posttranslationally glycosylated. Consequently, the carbohydrate side chains may have been attached to the EPO during production in mammalian, especially human, insect or yeast cells, which may be cells of a transgenic animal, either extracted from the animal or still in the animal.

These carbohydrate side chains may have been chemically or enzymatically modified after the expression in the appropriate cells, e.g. by removing or adding one or more carbohydrate moieties (see e.g. Dittmar, Conradt, Hauser, Hofer, Lindemaier, 1989, Advances in Protein design; Bloecker, Collins, Schmidt, and Schomburg eds., GBF-Monographs, 12, 231-246, VCH Publishers, Weinheim, N.Y., Cambridge)

According to an especially preferred embodiment of the present invention, the carbohydrate moiety is the terminal moiety of the carbohydrate side chain.

Consequently, in a preferred embodiment, the HES reacted with the crosslinking compound is linked to carbohydrate chains linked to N- and/or O-glycosylation sites of EPO.

It is also included within the present invention that the EPO contains a further carbohydrate moiety or further carbohydrate moieties to which the hydroxylamino group of a crosslinking compound is linked to. Techniques for attaching carbohydrate moieties to polypeptides, either enzymatically or by genetic engineering, followed by expression in appropriate cells, are known in the art (Berger, Greber, Mosbach, 1986, Galactosyltransferase-dependent sialylation of complex and endo-N-acetylglucosaminidase H-treated core N-glycans in vitro, FEBS Lett., 203(1), 64-8; Dittmar, Conradt, Hauser, Hofer, Lindenmaier, 1989, Advances in Protein design; Bloecker, Collins, Schmidt, and Schomburg eds., GBF-Monographs, 12, 231-246, VCH Publishers, Weinheim, N.Y., Cambridge).

In a preferred embodiment of the method of the invention, the carbohydrate moiety is oxidized in order to be able to react with the hydroxylamino group. This oxidation can be performed either chemically or enzymatically.

Methods for the chemical oxidation of carbohydrate moieties of polypeptides are known in the art and include the treatment with periodate (Chamow et al., 1992, J. Biol. Chem., 267, 15916-15922).

By chemically oxidizing, it is principally possible to oxidize any carbohydrate moiety, being terminally positioned or not. However, by choosing mild conditions (e.g., 1 mM periodate, 0° C. in contrast to harsh conditions, e.g.: 10 mM periodate 1 h at room temperature), it is possible to preferably oxidize the terminal sialic acid of a carbohydrate side chain.

Alternatively, the carbohydrate moiety may be oxidized enzymatically. Enzymes for the oxidation of the individual carbohydrate moieties are known in the art, e.g. in the case of galactose the enzyme is galactose oxidase.

If it is intended to oxidize terminal galactose moieties, it will be eventually necessary to partially or completely remove terminal sialic acids if the EPO has been produced in cells capable of attaching sialic acids to carbohydrate chains, e.g. in mammalian cells or in cells which have been genetically modified to be capable of attaching sialic acids to carbohydrate chains. Chemical or enzymatic methods for the removal of sialic acids are known in the art (Chaplin and Kennedy (eds.), 1996, Carbohydrate Analysis: a practical approach, especially Chapter 5 Montreuill, Glycoproteins, pages 175-177; IRL Press Practical approach series (ISBN 0-947946-44-3)).

However, it is also included within the present invention that the carbohydrate moiety to which the hydroxylamino group is linked to is suitably attached to the EPO. In the case it is preferred to attach galactose. This can be achieved by the means of galactosyltransferase. The methods are known in the art (Berger, Greber, Mosbach, 1986, Galactosyltransferase-dependent sialylation of complex and endo-N-acetylglucosaminidase H-treated core N-glycans in vitro, FEBS Lett., 203(1), 64-8).

In a most preferred embodiment of the present invention, at least one terminal saccharide unit of the EPO is oxidized, preferably galactose, most preferably sialic acid, of the one or more carbohydrate side chains of the EPO, optionally after partial or complete (enzymatic and/or chemical) removal of the terminal sialic acid, if necessary.

Consequently, the modified HES is conjugated to the oxidized terminal saccharide unit of the carbohydrate chain, preferably sialic acid.

Furthermore, the modified HES may be preferably conjugated to a terminal sialic acid, which is still more preferably oxidized.

Therefore, the present invention also relates to a conjugate and a method as described above, wherein the reaction is carried out at a temperature of from 20 to 25° C.

Therefore, the present invention also relates to a conjugate and a method as described above, wherein the carbohydrate moiety is an oxidized terminal saccharide unit of a carbohydrate side chain the erythropoietin, preferably an oxidized sialic acid.

Therefore, the present invention also relates to a conjugate and a method as described above, wherein the terminal saccharide unit was oxidized after partial or complete, enzymatic and/or chemical removal of the terminal sialic acid.

Therefore, the present invention also relates to a conjugate and a method as described above, wherein the terminal saccharide unit is galactose.

Therefore, the present invention also relates to a conjugate and a method as described above, wherein the carbohydrate moiety is comprised in a carbohydrate side chain of the erythropoietin which was attached to the erythropoietin via N- and/or O-linked glycosylation during its production in mammalian, especially human cells, insect cells, or yeast cells.

According to the present invention, a hydroxylamino group of a crosslinking compound is covalently linked to the carbohydrate moiety of EPO.

The term “hydroxylamino group” as used in the context of the present invention relates to a functional group according to formula —O—NH—R or —NH—O—R where R is hydrogen or an optionally suitably substituted alkyl residue, aryl residue, alkaryl residue or aralky residue. In case the hydroxylamino group —O—NH—R, R is preferably hydrogen and alkyl such as methyl, ethyl, propyl and butyl, more preferably hydrogen and methyl an especially preferably hydrogen. In case the hydroxylamino group —O—NH—R, R is preferably hydrogen and alkyl such as methyl, ethyl, propyl and butyl, more preferably hydrogen and methyl an especially preferably methyl. According to an especially preferred embodiment of the present invention, the hydroxylamino group —O—NH—R and R is hydrogen.

The crosslinking compound according to the invention may comprise the same or different hydroxlyamino groups, preferably the same hydroxylamino groups such as two methylaminooxy groups or two aminooxy groups or two methoxyamino groups.

The two hydroxylamino groups, comprised in the crosslinking compound according to the present invention, may be separated by a suitable spacer. Among others, the spacer may be an optionally substituted, linear, branched and/or cyclic hydrocarbon residue. Generally, the hydrocarbon residue has up to 60, preferably up to 40, more preferably up to 20, more preferably up to 10, more preferably up to 6 and especially preferably up to 4 carbon atoms. If heteroatoms are present, the spacer comprises generally from 1 to 20, preferably from 1 to 8, more preferably 1 to 6, more preferably 1 to 4 and especially preferably from 1 to 2 heteroatoms. As heteroatom, S, N or O are preferred, O being especially preferred. The hydrocarbon residue may comprise an optionally branched alkyl chain or an aryl group or a cycloalkyl group having, e.g., from 5 to 7 carbon atoms, or be an aralkyl group, an alkaryl group where the alkyl part may be a linear and/or cyclic alkyl group. According to an even more preferred embodiment of the present invention, the hydroxylamino groups are separated by a linear hydrocarbon chain having 4 carbon atoms. According to another preferred embodiment of the present invention, the functional groups are separated by a linear hydrocarbon chain having 4 carbon atoms and at least one, preferably one heteroatom, particularly preferably an oxygen atom. Particularly preferred is a spacer according to formula —CH₂—CH₂—O—CH₂—CH₂₋.

Therefore, preferred crosslinking compounds according to the present invention are

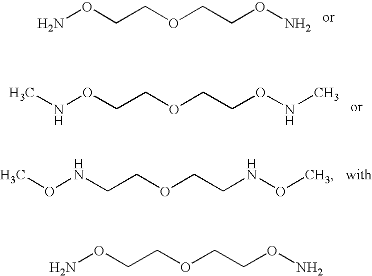

being especially preferred.

The conjugate of the present invention may exhibit essentially the same in-vitro biological activity as recombinant native EPO, since the in-vitro biological activity only measures binding affinity to the EPO receptor. Methods for determining the in-vitro biological activity are known in the art (see, e.g., Fibi et al., 1991, Blood, 77, 1203 ff; Kitamura et al, 1989, J. Cell Phys., 140, 323-334).

Furthermore, the conjugate of the present invention may exhibits a greater in vivo activity than the EPO used as a starting material for conjugation (non-conjugated EPO). Methods for determining the in vivo biological activity are known in the art (see, e.g., Example above). Furthermore, assays for the determination of in vivo activity are given in Example 7.

The conjugate may exhibit an in vivo activity of 10 to 500%, preferably 200 to 500%, more preferably 300% to 500%, more preferred 400% to 500% such as 45°% to 500% or 45° to 490% or 45°% to 480% or 45°% to 470%, the in vivo activity of the non-modified EPO set as 100%.

The high in vivo biological activity of the conjugate according to the present invention mainly results from the fact that the conjugate remains longer in the circulation than the non-conjugated EPO, because it is less recognized by the removal systems of the liver and because renal clearance is reduced due to the higher molecular weight. Methods for the determination of the in vivo half life time of EPO in the circulation are known in the art (Sytkowski, Lunn, Davis, Feldman, Siekman, 1998, Human erythropoietin dimers with markedly enhanced in vivo activity, Proc. Natl. Acad. Sci. USA, 95(3), 1184-8).

Consequently, it is a great advantage of the present invention that a conjugate is provided that may be administered less frequently than the EPO preparations commercially available at present. While standard EPO preparations have to be administered at least every 3 days, the conjugate of the invention is preferably administered twice a week, more preferably once a week.

According to the first step of the method of the present invention, HES is reacted with a hydroxylamino group, preferably with the group —O—NH₂ of the crosslinking compound.

In general, it is possible to react the hydroxylamino group with any suitable functional group of HES. According to especially preferred embodiments of the present invention, the hydroxylamino group is reacted with the reducing end of HES, which is oxidized or which is not oxidized.

In case the hydroxylamino group is reacted with the reducing end of HES in its oxidized form, HES is preferably used having a structure according to formula (IIa) 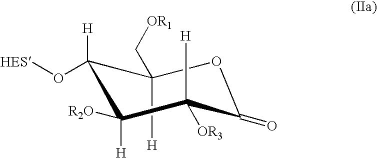
and/or according to formula (IIb)

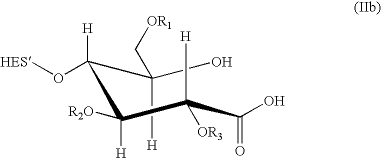

The oxidation of the reducing end of the polymer, preferably hydroxyethyl starch, may be carried out according to each method or combination of methods which result in compounds having the above-mentioned structures (IIa) and/or (IIb). This oxidation is preferably carried out using an alkaline iodine solution as described, e.g., in DE 196 28 705 A1 the respective contents of which (example A, column 9, lines 6 to 24) is incorporated herein by reference.

According to an especially preferred embodiment of the present invention, HES is employed with its reducing end in the non-oxidized form, i.e. HES according to formula (I).

Therefore, the present invention also relates to a method and a conjugate as described above, wherein the crosslinking compound is reacted with HES, HES being employed with its reducing end in the non-oxidized form.

It is possible to react the crosslinking compound with HES in any suitable solvent. According to an especially preferred embodiment, this reaction is carried out in an aqueous medium.

Therefore, the present invention also relates to a method and a conjugate as described above, wherein the hydroxyethyl starch is reacted with the homobifunctional crosslinking compound in an aqueous medium.

The term “aqueous medium” as used in the context of the present invention relates to a solvent or a mixture of solvents comprising water in the range of from at least 10% per weight, more preferably at least 20% per weight, more preferably at least 30% per weight, more preferably at least 40% per weight, more preferably at least 50% per weight, more preferably at least 60% per weight, more preferably at least 70% per weight, more preferably at least 80% per weight, even more preferably at least 90% per weight or up to 100% per weight, based on the weight of the solvents involved. The preferred reaction medium is water.

According to a particularly preferred embodiment, the present invention also relates to a method and a conjugate as described above, wherein HES is reacted with the crosslinking compound, preferably with the hydroxylamino group —O—NH₂ of the crosslinking compound, in an aqueous medium, and wherein the reducing end of HES is not oxidized prior to this reaction.

The pH of the reaction medium the reaction of HES and the crosslinking compound is carried out in is preferably in the range of from 4.5 to 6.5, more preferably in the range of from 5.0 to 6.0 and still more preferably in the range of from 5.0 to 5.5 such as at a pH of 5.0, 5.1, 5.2, 5.3, 5.4 or 5.5.

The pH may be adjusted to the above-mentioned values with any suitably buffer such as, e.g., an acetate buffer such as a sodium acetate buffer.

Therefore, the present invention also relates to a method and a conjugate as described above, wherein the reaction of HES and the crosslinking compound is carried out at a pH of from 4.5 to 6.5.

The temperature at which this reaction is carried out is generally in the range of from 5 to 30° C., preferably in the range of from 10 to 30° C., more preferably in the range of from 15 to 30° C., more preferably in the range of from 20 to 25° C. such as at a temperature of 20° C., 21° C., 22° C., 23° C., 24° C., or 25° C.

Therefore, the present invention also relates to a method and a conjugate as described above, wherein the reaction of HES and the crosslinking compound is carried out at a temperature of from 20 to 25° C.

Therefore, the present invention also relates to a method and a conjugate as described above, wherein the reaction of HES and the crosslinking compound is carried out at a pH of from 5.0 to 5.5 and at a temperature of from 20 to 25° C. in an aqueous medium, and wherein the reducing end of HES is not oxidized prior to this reaction.

According to the present invention, the HES derivative resulting from the reaction of HES with the crosslinking compound is reacted with the carbohydrate moiety of EPO.

It is possible to react the HES derivative compound with the carbohydrate moiety in any suitable solvent. According to an especially preferred embodiment, this reaction is carried out in an aqueous medium.

As to the term “aqueous medium”, reference is made to the definition given above.

Therefore, the present invention also relates to the method and conjugate as described above, wherein the hydroxylamino functionalized hydroxyethyl starch derivative is reacted with the carbohydrate moiety of the erythropoietin in an aqueous medium.

The pH of the reaction medium the reaction of HES derivative and the carbohydrate moiety of EPO is carried out in is preferably in the range of from 4.5 to 6.5, more preferably in the range of from 5.0 to 6.0 and still more preferably in the range of from 5.2 to 5.8 such as at a pH of 5.2, 5.3, 5.4, 5.5, 5.6, 5.7, or 5.8, particularly preferred at about 5.5

The pH may be adjusted to the above-mentioned values with any suitably buffer such as, e.g., an acetate buffer such as a sodium acetate buffer.

Therefore, the present invention also relates to a method and a conjugate as described above, wherein the reaction of the HES derivative and the carbohydrate moiety of EPO is carried out at a pH of from 4.5 to 6.5.

The temperature at which this reaction is carried out is generally in the range of from 5 to 30° C., preferably in the range of from 10 to 30° C., more preferably in the range of from 15 to 30° C., more preferably in the range of from 20 to 25° C. such as at a temperature of 20° C., 21° C., 22° C., 23° C., 24° C., or 25° C.

Therefore, the present invention also relates to a method and a conjugate as described above, wherein the reaction of the HES derivative and the carbohydrate moiety of EPO is carried out at a temperature of from 20 to 25° C.

Therefore, the present invention also relates to a method and a conjugate as described above, wherein the reaction of the HES derivative and the carbohydrate moiety of EPO is carried out at a pH of from 5.0 to 6.0 and at a temperature of from 20 to 25° C. in an aqueous medium.

According to an especially preferred embodiment, the present invention relates to a method wherein HES is reacted in an aqueous medium with a hydroxylamino group —O—NH₂ of a crosslinking compound in an aqueous medium to give a hydroxylamino functionalized HES derivative comprising an oxime linkage between HES residue and crosslinking compound residue, said method further comprising reacting the hydroxylamino functionalized HES derivative with a carbohydrate moiety of EPO, said carbohydrate moiety of EPO preferably being an oxidized terminal saccharide unit of a carbohydrate side chain of the EPO, more preferably an oxidized galactose residue and most preferably an oxidized sialic acid residue, to give a conjugate additionally comprising an oxime linkage between HES derivative residue and EPO.

Thus, according to one embodiment of the present invention where the crosslinking compound is reacted with the non-oxidized reducing end of HES, a conjugate 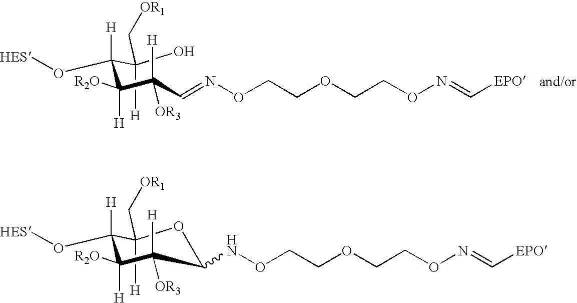
is obtained. The abbreviation EPO′ refers to the EPO molecule used for the reaction without the carbon atom of the carbohydrate moiety which is part of oxime linkage in the N═C double bond. The two structures above describe a structure where the crosslinking compound is linked via an oxime linkage to the reducing end of HES where the terminal saccharide unit of HES is present in the open form, and a structure with the respective cyclic aminal form where the crosslinking compound is linked to the reducing end of HES via an oxyamino group and where the terminal saccharide unit of HES is present in the cyclic form. Both structures may be simultaneously present in equilibrium with each other.

Therefore, the present invention also relates to a conjugate comprising hydroxyethyl starch, a crosslinking compound and erythropoietin, wherein the crosslinking compound is linked via an oxime linkage and/or an oxyamino group to the hydroxyethyl starch and via an oxime linkage to the carbohydrate moiety of the erythropoietin, and wherein the hydroxyethyl starch has a mean molecular weight of at least 40 kD and a degree of substitution of at least 0.6.

According to a further aspect of the present invention, a method is provided of how to improve the in vivo activity of HES-EPO conjugates by specifically changing the characteristics of the HES used for preparing the conjugate.

Therefore, the present invention also describes a method for increasing the specific in vivo activity of a second conjugate of erythropoietin and hydroxyethyl starch compared to a first conjugate of erythropoietin and hydroxyethyl starch by using two different hydroxyethyl starches for preparing these conjugates, wherein the hydroxyethyl starch used for the preparation of the second conjugate has an increased mean molecular weight and simultaneously an increased degree of substitution DS compared to the hydroxyethyl starch used for the preparation of the first conjugate.

According to a preferred embodiment of the present invention, this method specifically applies to improving the specific in vivo activity of conjugates of erythropoietin and hydroxyethyl starch by increasing the mean molecular weight of HES from about 10 kD to at least about 40 kD, preferably to at least about 50 kD, and more preferably to about 50 kD, and simultaneously increasing the degree of substitution DS from about 0.4 to at least about 0.6, more preferably to at least about 0.7, more preferably to about 0.7 or 0.8.

Therefore, the present invention also describes a method for improving the specific in vivo activity of conjugates of erythropoietin and hydroxyethyl starch as described above, wherein the mean molecular weight is increased from about 10 kD to at least about 40 kD, preferably to about 50 kD, and the degree of substitution of the hydroxyethyl starch is increased from about 0.4 to at least about 0.6, preferably to about 0.7 to 0.8.

Therefore, e.g., in order to improve the specific in vivo activity of conjugates of erythropoietin and hydroxyethyl starch, a conjugate comprising hydroxyethyl starch having a mean molecular weight of about 10 kD and a DS of about 0.4 should be replaced by a conjugate comprising hydroxyethyl starch having, e.g., a mean molecular weight of about 50 kD and a DS of about 0.7 to about 0.8.

It is believed that this method applies to many HES-EPO conjugates, especially to HES-EPO conjugates in which HES and EPO are covalently linked via at least one crosslinking compound, particularly via one crosslinking compound which is preferably linked to HES by reacting a hydroxylamino group of the crosslinking compound with HES, most preferably in an aqueous medium, and by reacting a further hydroxylamino group of the crosslinking compound with EPO, most preferably in an aqueous medium, more preferably with a carbohydrate moiety of EPO, still more preferably with a carbohydrate moiety preferably being an oxidized terminal saccharide unit of a carbohydrate side chain of EPO such as an oxidized galactose residue or sialic acid residue.

Therefore, the present invention also relates to the method for improving the specific in vivo activity of conjugates of erythropoietin and hydroxyethyl starch as described above, wherein the conjugate comprises a crosslinking compound having two hydroxylamino groups, one of which is covalently linked to a carbohydrate moiety of the erythropoietin and one of which is covalently linked to the hydroxyethyl starch.

In the methods for preparing a conjugate of the invention the conversion rate in the above described methods may be at least 50%, more preferred at least 70%, even more preferred at least 80% and in particular 95% or even more, such as at least 98% or 99%.

Moreover, it is believed that this method for improving the specific in vivo activity of conjugates of erythropoietin and hydroxyethyl starch may also apply to other proteins and other hydroxyalkyl starches such as hydroxypropyl starches and hydroxybutyl starches. Examples of proteins are, e.g., Examples of other proteins are, e.g., colony-stimulating factors (CSF), such as G-CSF or GM-CSF like recombinant human G-CSF or GM-CSF (rhG-CSF or rhGM-CSF), alpha-Interferon (IFN alpha), beta-Interferon (IFN beta) or gamma-Interferon (IFN gamma), such as IFN alpha and IFN beta like recombinant human IFN alpha or IFN beta (rhIFN alpha or rhIFN beta), interleukines, e.g. IL-1 to IL-18 such as IL-2 or IL-3 like recombinant human IL-2 or IL-3 (rhIL-2 or rhIL-3), serum proteins such as coagulation factors II-XIII like factor VIII, alpha1-antitrypsin (A1AT), activated protein C (APC), plasminogen activators such as tissue-type plasminogen activator (tPA), such as human tissue plasminogen activator (hTPA), AT III such as recombinant human AT m (rhAT III), myoglobin, albumin such as bovine serum albumin (BSA), growth factors, such as epidermal growth factor (EGF), thrombocyte growth factor (PDGF), fibroblast growth factor (FGF), brain-derived growth factor (BDGF), nerve growth factor (NGF), B-cell growth factor (BCGF), brain-derived neurotrophic growth factor (BDNF), ciliary neurotrophic factor (CNTF), transforming growth factors such as TGF alpha or TGF beta, BMP (bone morphogenic proteins), growth hormones such as human growth hormone, tumor necrosis factors such as TNF alpha or TNF beta, somatostatine, somatotropine, somatomedines, hemoglobin, hormones or prohormones such as insulin, gonadotropin, melanocyte-stimulating hormone (alpha-MSH), triptorelin, hypthalamic hormones such as antidiuretic hormones (ADH and oxytocin as well as releasing hormones and release-inhibiting hormones, parathyroid hormone, thyroid hormones such as thyroxine, thyrotropin, thyroliberin, prolactin, calcitonin, glucagon, glucagon-like peptides (GLP-1, GLP-2 etc.), exendines such as exendin-4, leptin, vasopressin, gastrin, secretin, integrins, glycoprotein hormones (e.g. LH, FSH etc.), melanoside-stimulating hormones, lipoproteins and apo-lipoproteins such as apo-B, apo-E, apo-La, immunoglobulins such as IgG, IgE, IgM, IgA, IgD and fragments thereof, hirudin, tissue-pathway inhibitor, plant proteins such as lectin or ricin, bee-venom, snake-venom, immunotoxins, antigen E, alpha-proteinase inhibitor, ragweed allergen, melanin, oligolysine proteins, RGD proteins or optionally corresponding receptors for one of these proteins; or a functional derivative or fragment of any of these proteins or receptors.

According to another aspect, the present invention also relates to method for screening for a conjugate of erythropoietin and hydroxyalkyl starch, preferably hydroxyethyl starch, having improved in vivo activity compared to native erythropoietin comprising the steps of

(i) providing a candidate conjugate;

(ii) testing the in vivo activity in comparison with native erythropoietin,

wherein the mean molecular weight MW is varied in the range of from 1 to 300 kD and the degree of substitution DS is varied in the range of from 0.1 to 1.0, and wherein these parameters are simultaneously increased compared to a given combination of parameters.

According to a preferred embodiment, the given combination of parameters is a mean molecular weight MW of about 10 kD and a degree of substitution DS of about 0.4.

Therefore, the present invention also relates to the screening method as described above, wherein the given combination of parameters is a mean molecular weight MW of about 10 kD and a degree of substitution DS of about 0.4. According to a further aspect, the present invention also relates to the screening method as described above, said method further comprising the step of incorporating the candidate conjugate into a therapeutic or prophylactic composition.

According to yet another aspect, the present invention also relates to a conjugate as described above or a conjugate, obtainable by a method as described above, for use in a method for the treatment of the human or animal body.

The conjugates according to the invention may be at least 50% pure, even more preferred at least 70% pure, even more preferred at least 90%, in particular at least 95% or at least 99% pure. In a most preferred embodiment, the conjugates may be 100% pure, i.e. there are no other by-products present.

Therefore, according to another aspect, the present invention also relates to a composition which may comprise the conjugate(s) of the invention, wherein the amount of the conjugate(s) may be at least 50 wt-%, even more preferred at least 70 wt-%, even more preferred at least 90 wt-%, in particular at least 95 wt.-% or at least 99 wt.-%. In a most preferred embodiment, the composition may consist of the conjugate(s), i.e. the amount of the conjugate(s) is 100 wt.-%.

Accordingly, the present invention relates to a pharmaceutical composition comprising in a therapeutically effective amount a conjugate as described above or a conjugate, obtainable by a method as described above.

The term “therapeutically effective amount” as used in the context of the present invention relates to that amount which provides therapeutic effect for a given condition and administration regimen.

Moreover, the present invention relates to a pharmaceutical composition as described above, further comprising at least one pharmaceutically acceptable diluent, adjuvant, or carrier.

According to another aspect, the present invention also relates to the use of a conjugate as described above or a HES-EPO conjugate, obtainable by a method as described, for the preparation of a medicament for the treatment of anemic disorders or hematopoietic dysfunction disorders or diseases related thereto. The invention further relates to the use of a HES-EPO conjugate as described above or a HES-EPO conjugate, obtainable by a method as described above, for the preparation of a medicament for the treatment of anemic disorders or hematopoietic dysfunction disorders or diseases related hereto.

The administration of erythropoietin isoforms is preferably by parenteral routes. The specific route chosen will depend upon the condition being treated. The administration of erythropoietin isoforms is preferably done as part of a formulation containing a suitable carrier, such as human serum albumin, a suitable diluent, such as a buffered saline solution, and/or a suitable adjuvant. The required dosage will be in amounts sufficient to raise the hematocrit of patients and will vary depending upon the severity of the condition being treated, the method of administration used and the like. The object of the treatment with the pharmaceutical composition of the invention is preferably an increase of the hemoglobin value of more than 6.8 mmol/l in the blood. For this, the pharmaceutical composition may be administered in a way that the hemoglobin value increases between 0.6 mmol/l and 1.6 mmol/l per week. If the hemoglobin value exceeds 8.7 mmol/l, the therapy should be preferably interrupted until the hemoglobin value is below 8.1 mmol/l. The composition of the invention is preferably used in a formulation suitable for subcutaneous or intravenous or parenteral injection. For this, suitable excipients and carriers are e.g. sodium dihydrogen phosphate, disodium hydrogen phosphate, sodium chlorate, polysorbate 80, HSA and water for injection. The composition may be administered three times a week, preferably two times a week, more preferably once a week, and most preferably every two weeks. Preferably, the pharmaceutical composition is administered in an amount of 0.01-10 μg/kg body weight of the patient, more preferably 0, 1 to 5 μg/kg, 0, 1 to 1 μg/kg, or 0.2-0.9 μg/kg, most preferably 0.3-0.7 μg/kg, and most preferred 0.4-0.6 μg/kg body weight. In general, preferably between 10 μg and 200 μg, preferably between 15 μg and 100 μg are administered per dosis.

Accordingly, the present invention also relates to the use of a conjugate as described above or a conjugate, obtainable by a method as described above, for the preparation of a medicament for the treatment of anemic disorders or hematopoietic dysfunction disorders or diseases related thereto.

The invention will be further described in the following examples, which do not limit the scope of the invention described in the claims.

### EXAMPLES

Sample Code HES-Modification of EPO

0312-17/A14: EPO starting material, not modified

0401-09/A20: HES 10/0.4; periodate oxidised

0401-09/A21: HES 10/0.7; periodate oxidised

0401-09/A23: HES 50/0.7; periodate oxidised

0401-09/A24: A14/mock-incubated with unmodified HES; periodate oxidised

0401-09/A25: HES 50/0.4; periodate oxidised

Sample identification (short form) e.g. for the starting material is A14; A20=periodate oxidised EPO A14 after modification with HES 10/04.

### Example 1

### Periodate oxidation of N-acetylaneuraminic acid residues by mild periodate treatment of EPO

To a 2,0 mg/ml solution of EPO (recombinantly produced EPO having amino acid sequence of human EPO and similar or essentially the same characteristics as the commercially available Epoietin alpha: Erypo, ORTHO BIOTECH, Jansen-Cilag or Epoietin beta: NeoRecormon, Roche; cf. EP 0 148 605, EP 0 205 564, EP 0 411 678) of total 20 ml kept at 0° C. were added 2,2 ml of an ice-cold solution of 1 mM sodium meta-periodate resulting in a final concentration of 1 mM sodium meta-periodate. The mixture was incubated at 0° C. for 1 hour in an ice-bath in the dark and the reaction was terminated by addition of 40 μl of glycerol and incubated for further 5 minutes.

### Example 2

### Buffer Exchange of Periodate Oxidised EPO for Subsequent Derivatisation with a Hydroxylamino Functionalized Hydroxyethyl Starch Derivative

Buffer exchange was performed using a 20 ml Vivaspin 20 concentrator (Vivaspin AG, Hannover, Germany) with a polyethersulfone (PES) membrane and a molecular weight cut-off 10 kD. First, the concentrator unit was washed by addition of5 ml of 0.1 M Na-acetate buffer pH 5.5 and centrifugation of the concentrator unit at 4000 rpm at 6° C. in a Megafuge 1.0R (Kendro Laboratory Equipment, Osterode, Germany). Subsequently, 20 ml of the periodate oxidised EPO solution according to Example 1 was added to the concentrator unit and was centrifuged at 4000 rpm for 25 min until a 5-fold concentration was achieved. 15 ml of 0.1 M Na-acetate buffer pH 5.5 was added to the concentrate and then centrifuged as described above. The centrifugation cycle was repeated 3 times, the final concentrate was removed and transferred into a 50 ml sterile plastic tube, after washing of the concentrator unit 2 times with each 1 ml of Na-acetate buffer pH 5.5; the volume of the EPO solution was adjusted with Na-acetate buffer pH 5.5 to 26.7 ml and protein concentration of the final oxidised EPO solution was determined by measuring the absorbance at 280 nm using the specific absorbance value of 7.43 as described in the European Pharmacopeia (Erythropoietin Concentrated Solution, 4th Edition, 2002, pages 1123-1128). A value of 1.378 mg/ml was determined for the final periodate oxidised EPO solution (36.8 mg EPO, corresponding to ˜90% final yield).

### Example 3

### Synthesis of Conjugates of Hydroxyethyl Starch and EPO

### Example 3.1

### Synthesis of Hydroxylamino Functionalized HES Derivatives

O-[2-(2-aminooxy-ethoxy)-ethyl]-hydroxylamine was synthesized as described in Boturyn et al. Tetrahedron 53 (1997) p. 5485-5492 in 2 steps from commercially available materials.

### Example 3.1(a)

### Synthesis of Hydroxylamino-HES 10/0.4

2 g of HES10/0.4 (MW=10000 D, DS=0.4, Supramol Parenteral Colloids GmbH, Rosbach-Rodheim, D) were dissolved in 17 mL 0.1M sodium acetate buffer, pH 5.2 and 20 mmol O-[2-(2-aminooxy-ethoxy)-ethyl]-hydroxylamine were added. After shaking for 19 h at 22° C., the reaction mixture was added to 100 mL of an ice-cold 1:1 mixture of acetone and ethanol (v/v). The precipitated product was collected by centrifugation at 4° C., re-dissolved in 50 mL water, dialysed for 21 h against water (SnakeSkin dialysis tubing, 3.5 kD cut off, Perbio Sciences Deutschland GmbH, Bonn, D) and lyophilized.

The molecular weight of the HES10/0.4 when measured with LALLS-GPC was 8.4 kD and the DS was 0.41.

### Example 3.1 (b)

### Synthesis of Hydroxylamino-HES 10/0.7

2 g of HES 10/0.7 (MW=10000 D, DS=0.7, Supramol Parenteral Colloids GmbH, Rosbach-Rodheim, D) were dissolved in 18 mL 0.1M sodium acetate buffer, pH 5.2 and 20 mmol O-[2-(2-aminooxy-ethoxy)-ethyl]-hydroxylamine were added. After shaking for 19 h at 22° C., the reaction mixture was added to 100 mL of an ice-cold 1:1 mixture of acetone and ethanol (v/v). The precipitated product was collected by centrifugation at 4° C., re-dissolved in 50 mL water, dialysed for 21 h against water (SnakeSkin dialysis tubing, 3.5 kD cut off, Perbio Sciences Deutschland GmbH, Bonn, D) and lyophilized.

The molecular weight of the HES 10/0.7 when measured with LALLS-GPC was 10.5 kD and the DS was 0.76.

### Example 3.1(c)

### Synthesis of Hydroxylamino-HES 50/0.4

2 g of HES50/0.4 (MW=50000 D, DS=0.4, Supramol Parenteral Colloids GmbH, Rosbach-Rodheim, D) were dissolved in 20 mL 0.1M sodium acetate buffer, pH 5.2 and 4 mmol O-[2-(2-aminooxy-ethoxy)-ethyl]-hydroxylamine were added. After shaking for 19 h at 22° C., the reaction mixture was added to 100 mL of an ice-cold 1:1 mixture of acetone and ethanol (v/v). The precipitated product was collected by centrifugation at 4° C., re-dissolved in 50 mL water, dialysed for 21 h against water (SnakeSkin dialysis tubing, 3.5 kD cut off, Perbio Sciences Deutschland GmbH, Bonn, D) and lyophilized.

The molecular weight of the HES50/0.4 when measured with LALLS-GPC was 55.7 kD and the DS was 0.41.

### Example 3.1 (d)

### Synthesis of Hydroxylamino-HES 50/0.7

2 g of HES50/0.7 (MW=50000 D, DS=0.7, Supramol Parenteral Colloids GmbH, Rosbach-Rodheim, D) were dissolved in 20 mL 0.1M sodium acetate buffer, pH 5.2 and 4 mmol O-[2-(2-aminooxy-ethoxy)-ethyl]-hydroxylamine were added. After shaking for 17.5 h at 22° C., the reaction mixture was added to 70 mL of an ice-cold 1:1 mixture of acetone and ethanol (v/v). The precipitated product was collected by centrifugation at 0° C., washed with 30 mL of an ice-cold 1:1 mixture of acetone and ethanol (v/v), re-dissolved in 50 mL water, dialysed for 19.5 h against water (SnakeSkin dialysis tubing, 3.5 kD cut off, Perbio Sciences Deutschland GmbH, Bonn, D) and lyophilized.

The molecular weight of the HES50/0.7 when measured with LALLS-GPC was 46.9 kD and the DS was 0.76.

### Example 3.2

### Synthesis of HES-EPO Conjugates

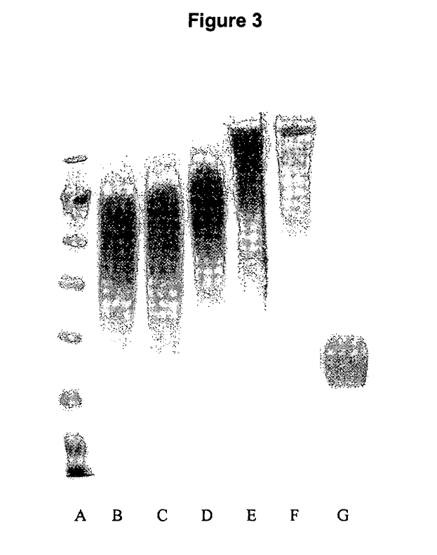

In Examples 3.2(a) to 3.2(d), a successful conjugation is indicated by the migration of the protein bands to higher molecular weights in the SDS page analysis according to FIG. 3. The increased band-with is due to the molecular weight distribution of the HES derivatives used and the number of HES derivatives linked to the protein.

### Example 3.2(a)

### Synthesis with of Hydroxylamino-HES 10/0.4 According to Example 3.1(a)

To 3.63 mL of a solution of oxidized EPO in 0.1 M sodium acetate buffer, pH 5.5 (according to Example 2; 1.378 mg/ml), 83 mg of hydroxylaminoHES10/0.4, produced according to example 3.1 (a), were added and the solution was shaken for 16.5 h at 22° C.

### Example 3.2(b)

### Synthesis with of Hydroxylamino-HES 10/0.7 According to Example 3.1(b)

To 3.63 mL of a solution of oxidized EPO in 0.1 M sodium acetate buffer, pH 5.5 (according to Example 2; 1.378 mg/ml), 83 mg of hydroxylaminoHES 10/0.7, produced according to example 3.1(b), were added and the solution was shaken for 16.5 h at 22° C.

### Example 3.2(c)

### Synthesis with of Hydroxylamino-HES 50/0.4 According to Example 3.1(c)

To 3.63 mL of a solution of oxidized EPO in 0.1 M sodium acetate buffer, pH 5.5 (according to Example 2; 1.378 mg/ml), 416 mg of hydroxylaminoHES50/0.4, produced according to example 3.1(c), were added and the solution was shaken for 16.5 h at 22° C.

### Example 3.2(d)

### Synthesis with of Hydroxylamino-HES 50/0.7 According to Example 3.1(d)

To 3.63 mL of a solution of oxidized EPO in 0.1 M sodium acetate buffer, pH 5.5 (according to Example 2; 1.378 mg/ml), 416 mg of hydroxylaminoHES50/0.7, produced according to example 3.1(d), were added and the solution was shaken for 16.5 h at 22° C.

### Example 4

### Purification of HES-Modified EPO and Separation of Unreacted HES-Derivatives from HES-Modified EPO

Subsequent to the HES-coupling procedures according to Examples 3.2(a) to 3.2(d), the purification of all samples was performed at room temperature using an ÄKTA explorer 10 system equipped with a Pump P-903, Mixer M-925 with 0.6 ml chamber, Monitor pH/C-90°, pump P-950 (sample pump) along with a Software Unicorn Version 3.21. Detection was at 280, 260 and 220 nm using a Monitor UV-90° with a 10 mm flow cell.

The incubation mixtures were diluted with 10 volumes of buffer A (20 mM N-morpholino propane sulfonic acid adjusted to pH 8.0 with NaOH) and were applied to a column containing 4 ml Q-Sepharose Fast Flow (Amersham Pharmacia Biotech) at a flow rate of 0.8 ml/min; the column was previously equilibrated with 7 column volumes (CV) of buffer A. The column was then washed with 6 CV of buffer A at a flow rate of 1.0 ml/min and elution was performed by using 2.5 CV of buffer B (0.5 M NaCl in 20 mM Na-phosphate, pH 6.5) at a flow rate of 0.6 ml/min. The column was then washed with 2.5 CV of buffer C (1.5 M NaCl in 20 mM Na-phosphate, pH 6.5) at a flow rate of 0.6 ml/min and was re-equilibrated by passing 7 CV of buffer A at flow rate of 1.0 ml/min.

Samples from incubations with (activated) hydroxylaminoHES derivatives all yielded significant absorption at 220 nm. Samples A20 and A21 (incubated with hydroxylaminoHES10/0.4 and 10/0.7, respectively) gave no detectable absorption at 280 nm, whereas samples A23 and A25 (incubated with HydroxylaminoHES50/0.7 and 50/0.4, respectively) yielded 800 mAU×ml and 950 mAU×ml, respectively. The bound proteins were recovered in a volume of 6.5-8.0 ml almost exclusively in eluate 1, with eluate 2 containing <2% of the peak area of totally eluted peaks detected at 280 nm. The protein recovery was comparable for all EPO samples (approximately 85%).

HES-modified EPO and EPO from appropriate control incubations were subjected to buffer exchange by using 5 ml Vivaspin concentrators (10,000 MW cut-off) and centrifugation at 4000 rpm at 6° C. as described previously. Samples (1-3 mg of EPO protein) were concentrated to 0.5-0.7 ml and were diluted with phosphate buffered saline (PBS) pH 7.1 to 5 ml and subjected to 10-fold concentration by centrifugation. Each sample was subjected to the concentration and dilution cycle three times. Finally, samples were withdrawn and the concentrator units were washed with 2×0.5 ml of PBS. Samples were frozen in liquid nitrogen at protein concentrations of approximately 1.2 mg/ml.

### Example 5

### Analytical Experiments

### Example 5.1

### Liberation of N-Linked Oligosaccharides with Recombinant polypeptide N-glycosidase (Roche, Penzberg, Germany)

To 400-1.2 mg aliquots of native, periodated oxidised or HES-modified EPO in 50 mM Na-phosphate buffer pH 7.2 were added 4011 of recombinant polypeptide N-glycosidase (Roche, Penzberg, Germany; 250 units/250 μl lot: 101610420). The reaction mixture was incubated at 37° C. for 12-18 hours and the release of N-glycosidically bound oligosaccharides was checked by SDS-PAGE analysis of 5-10 μg protein under reducing conditions and subsequent staining of protein bands with Coomassie Blue (Carl Roth GmbH Karlsruhe, Germany) and detection of the specific shift of the EPO protein band to the migration position of the de-N-glycosylated EPO forms.

### Example 5.2

### Separation of N-linked Oligosaccharides from de-N-Glycosylated EPO Protein by RP-HPLC

Separation of all de-N-glycosylated EPO samples from HES-modified and unmodified EPO protein samples was performed at room temperature using an ÄKTA explorer 10 system equipped with a Pump P-903, Mixer M-925 with 0.6 ml chamber, Monitor pH/C-900, pump P-950 (sample pump) along with a Software Unicorn Version 3.21. Detection was at 280, 220 and 206 nm using a Monitor UV-900 with a 10 mm flow cell.

Runs were performed at room temperature using the ÄKTA explorer 10 equipment and flow rate of 4 ml/min. Aliquots of PNGase digests of 1.1-1.2 mg HESylated EPO were applied to a PepRPC 15 μm column (PepRPC 15 μm, 2 cm×10 cm; Pharmacia) which was equilibrated with 1.25 CV of 9% eluent B (0.1% TFA, 90% acetonitrile). 1.25 ml samples of de-N-glycosylated EPO forms were then injected and the sample loop was washed with 12 ml of 9% eluent B. Following washing of the column with 0.2 CV of 9% eluent B, a linear gradient from 9% to 90% eluent B over 2 CV was applied. Elution of the column was continued by using 0.5 CV of 90% eluent B, and finally the column was re-equilibrated with 1.0 CV of 9% eluent B. Fractions were collected every 1 min (4 ml).

The oligosaccharides were recovered from the flow through (fractions 1-3; 4 ml each fraction) and, in the case of HESylated EPO, from fractions 6-8 eluting at a concentration of about 20% eluent B. The protein eluted in a volume of 10-12 ml at a concentration of 54% eluent B. The recovery of the de-N-glycosylated EPO was comparable for all samples, yielding a mean value of 581 mAU×ml×mg⁻¹, with a relative standard deviation of 3.8%.

(a) The EPO protein fractions (containing EPO forms modified with HES at the O-glycan moiety) were diluted with 1 volume of water and were lyophilized. Subsequently the dried samples were re-solubilized in water and after neutralisation with NaOH were subjected to concentration using 5 ml Vivaspin concentrators.

(b) The oligosaccharide fractions (see above: fractions 1-3 combined with fractions 6-8, respectively) were concentrated in a speed-vac concentrator after neutralisation and were subsequently de-salted using Vivaspin 5 concentrators (cut-off 5000). The oligosaccharides were adjusted to a final volume of 0.5 ml and were frozen in liquid nitrogen and kept at −20° C.

(c) For analytical purposes, the released N-glycans were separated from the polypeptide by addition of 3 volumes of cold 100% ethanol and incubation at −20° C. for at least 2 hours. The precipitated protein was removed by centrifugation at 13,000 rpm for 10 minutes at 4° C. The pellet was then subjected to two additional washes with 500 μl ice-cold 70% ethanol. The oligosaccharides in the pooled supernatants were dried in a vacuum centrifuge (Speed Vac concentrator, Savant Instruments Inc., USA). The glycan samples were desalted using Hypercarb cartridges (100 or 200 mg) as follows: prior to use, the cartridges were washed three times with 500 μl 80% (v/v) acetonitrile in 0.1% (v/v) TFA followed by three washes with 500 μL water. The samples were diluted with water to a final volume of at least 300 μl before loading onto the cartridges. They were rigorously washed with water.

Oligosaccharides were eluted with 1.2 ml 25% acetonitrile containing 0.1% (v/v) TFA. The eluted oligosaccharides were neutralised with 2 M NH₄OH and were dried in a Speed Vac concentrator. They were stored at −20° C. in H₂O until further use.

### Example 5.3

### Oligosaccharid Analysis—Mild Acid Hydrolysis of Oligosaccharides (Removal of Sialic Acids and HES-Modified Sialic Acids from Oligosacharides)

Aliquots of the desalted oligosaccharides (compare 5.2(a)) were mixed with the same volume of 10 mM H₂SO₄ and were incubated for 90 minutes at 80° C. After neutralisation with 50 mM NaOH the desialylated glycans were dried in a speed-vac and were adjusted to an appropriate concentration for analysis in HPAEC-PAD (high-pH-anion exchange chromatography with pulsed amperometric detection).

### Example 5.4

### Oligosaccharide Mapping by HPAEC-PAD (high-pH-Anion Exchange Chromatography with Pulsed Amperometric Detection)

BioLC System, (Dionex, Sunnyvale) consisting of a AS50 Autosampler, AS50 Thermal Compartment, ED50 Electrochemical Detector, GS50 Gradient Pump, Software Chromeleon Chromatography Management System, was used along with a CarboPac PA-100 separation column (4×250 mm) and a CarboPac PA-100 pre-column (4×50 mm). Two different modes were used for the mapping and for quantitation of oligosaccharides.

### Example 5.4(a)

### HPAEC-PAD Asialo-Mode

Neutral oligosaccharides were subjected to HPAEC-PAD mapping using a gradient of solvent A (200 mM NaOH) and solvent B (200 mM NaOH plus 600 mM Na-acetate) as depicted in Table 1:

TABLE 1
Gradient for mapping of neutral oligosacharides

| Time [min] | solvent A [%] | solvent B [%] |
|---|---|---|
| 0 | 100 | 0 |
| 5 | 100 | 0 |
| 35 | 80 | 20 |
| 45 | 70 | 30 |
| 47 | 0 | 100 |
| 52 | 0 | 100 |
| 53 | 100 | 0 |
| 60 | 100 | 0 |

Flow rate: 1 ml/min

The detector potentials for the electrochemical detector were as shown in Table 2:

TABLE 2
Detector-Potentials for oligosaccharides

| Time [ms] | potential [mV] |
|---|---|
| 0 | 50 |
| 200 | 50 |
| 400 | 50 |
| 410 | 750 |
| 600 | 750 |
| 610 | −150 |
| 1000 | −150 |

### Example 5.4(b)

### HPAEC-PAD Oligos-Mode

Native oligosaccharides were subjected to HPAEC-PAD mapping using a gradient of solvent C (100 mM NaOH) and solvent D (100 mM NaOH plus 600 mM Na-Table 3:

TABLE 3
Gradient mapping of native (sialylated) oligosaccharides

| Time [min] | solvent C [%] | solvent D [%] |
|---|---|---|
| 0 | 100 | 0 |
| 2 | 100 | 0 |
| 50 | 65 | 35 |
| 60 | 0 | 100 |
| 63 | 0 | 100 |
| 64 | 100 | 0 |
| 70 | 100 | 0 |

Flow rate: 1 ml/min

The detector potentials for the electrochemical detector were as shown in Table 4:

TABLE 4
Detector-Potentials for oligosaccharides

| Time [ms] | Potential [mV] |
|---|---|
| 0 | 50 |
| 200 | 50 |
| 400 | 50 |
| 410 | 750 |
| 600 | 750 |
| 610 | −150 |
| 1000 | −150 |

The specific peak areas (nC×min×nmol⁻¹) were calculated using response factors obtained with defined oligosaccharide standards (disialylated diantennary, trisialylated triantennary, and terasialylated tetraantennary structures with and without N-acetyllactosamine repeats all containing proximal alpha-1,6-linked fucose (Nimtz et al., 1993, Schroeter et al., 1999, Grabenhorst et al., 1999).

### Example 5.5

### Mass Spectrometry of Peptides and Oligosaccharides

### Example 5.5(a)

### Analysis by Matrix-Assisted Laser Desorption/Ionization Time-of-Flight Mass Spectrometry (MALDI/TOF/TOF-MS

Intact glycoprotein preparations (chemically desialylated or enzymatically de-N-glycosylated forms) were analyzed with a Bruker ULTRAFLEX time-of-flight (TOF/TOF) instrument in the linear positive ion mode using a matrix of 22.4 mg 3,5 dimethoxy-4-hydroxy-cinnamic acid in 400 μl acetonitrile and 600 μl 0.1% (v/v) trifluoroacetic acid in H₂O; (glyco)-peptides were measured using a matrix of 19 mg alpha-cyano-4-hydroxycinnamic acid in the same solvent mixture using the reflectron for enhanced resolution. Native desialylated oligosaccharides were analyzed using 2,5-dihydroxybenzoic acid as UV-absorbing material in the positive as well as in the negative ion mode using the reflectron in both cases. For MS-MS analyses, selected parent ions were subjected to laser induced dissociation (LID) and the resulting fragment ions separated by the second TOF stage (LIFT) of the instrument. Sample solutions of 1 μl and an approximate concentration of 1-10 pmol·μl⁻¹ were mixed with equal amounts of the respective matrix. This mixture was spotted onto a stainless steel target and dried at room temperature before analysis.

### Example 5.5(b)

### Electrospray Ionisation MS

1-3 μl aliquots of the tryptic digests corresponding to 2-20 pmol of protein were applied to a nanospray gold-coated glass capillary placed orthogonally in front of the entrance hole of a QTOF-II instrument (Micromass, UK). 1000 V were applied to the capillary and ions were separated by the time-of-flight analyser. For MS/MS analysis parent ions were selected by the quadrupole mass filter and subjected to collision induced dissociation. Resulting daughter ions were then separated by the TOF-analyzer. Spectra were processed by the MaxEnt3-programme (Micromass, UK) and the peptide sequence was determined using the PepSeq software.

### Example 5.5(c)

### Compositional Analysis of Oligosaccharides from Untreated and HES-Modified EPO

Monosaccharides were analyzed as the corresponding methyl glycosides after methanolysis, N-reacetylation and trimethylsilylation by GC/MS [Chaplin, M. F. (1982) A rapid and sensitive method for the analysis of carbohydrate components in glycoproteins using gas-liquid chromatography; Anal Biochem. 1982 Jul. 1;123(2):336-41]. The analyses were performed on a Finnigan GCQ ion trap mass spectrometer (Finnigan MAT corp., San Jose, Calif.) running in the positive ion EI mode equipped with a 30 m DB5 capillary column. Temperature program: 2 min isotherm at 80° C., then 10 degrees min⁻¹ to 300° C.

Monosaccharides were identified by their retention time and characteristic fragmentation pattern. The uncorrected results of electronic peak integration were used for quantification. Monosaccharides yielding more than one peak due to anomericity and/or the presence of furanoid and pyranoid forms were quantified by adding all major peaks. 2.0 μg of myo-inositol was added to samples and was used as an internal standard.

### Example 6

### Preparation of Samples for Mouse Bioassay

HES-EPO conjugate samples, prepared according to Example 4 (2-3 mg/ml) were filtered through a 0.2 μm, Corning syringe filter unit (15 mm; RC membrane; Cat. No, 431215; Corning Incorporated, NY 14831). The samples were then frozen in liquid nitrogen in cryo vials and stored at −20° C. until further use (see Example 7). EPO protein concentration was determined by UV-absorbance measurement at 280 nm according to European Pharmacopoeia, Fourth Edition, 2002, Directorate for the Quality of Medicines of the Council of Europe (EDQM).

### Example 7

### In-Vivo Assay of the Biological Activity of HES-Modified EPO

The EPO-bioassay in the normocythaemic mouse system was performed according to the procedures described in the European Pharmacopeia 4, Monography 01/2002:1316 on the basis of the HES-EPO prepared according to Example 6: Erythropoietin concentrated solution and Ph. Eur. Chapter 5.3: “Statistical Analysis of Results of Biological Assays and Tests”; in deviation from this assay the laboratory that carried out the EPO assay was using the international BRP EPO reference standard preparation in a 4-fold dilution. Therefore it was necessary to divide the received results by 4.

For the HES-modified EPO 50/0.7 a value for the specific activity of 533 000 units per mg EPO of protein was measured indicating an approximately 4-5 fold higher specific activity when compared to the EPO starting material. The results of the study are summarized in Table 5:

TABLE 5

| | | | Calculated specific |
|---|---|---|---|
| | | | activity of EPO |
| | | | sample |
| | Value | Value/4 | (based on A280 and |
| | (normocythaemic | (normocythaemic | RP-HPLC |
| Sample description | mouse assay) | mouse assay) | determination) |
| EPO starting material | 354 | 89 | 115 900 U/mg |
| (not modified) | | | |
| EPO- | 178 | 45 | 117 000 U/mg |
| HydroxylaminoHES10/0.4 | | | |
| EPO- | 459 | 115 | 299 000 U/mg |
| HydroxylaminoHES10/0.7 | | | |
| EPO- | 228 | 57 | 149 700 U/mg |
| HydroxylaminoHES50/0.4 | | | |
| EPO starting material | 208 | 52 | 67 600 U/mg |
| (incubated with HES10/0.4; | | | |
| per-ox) | | | |
| EPO | 821 | 205 | 533 000 U/mg |
| HydroxylaminoHES50/0.7 | | | |

### RESULTS

### Results 1

### Purification of EPO and Modified EPO Forms by Anion Exchange Chromatography on Q-Sepharose

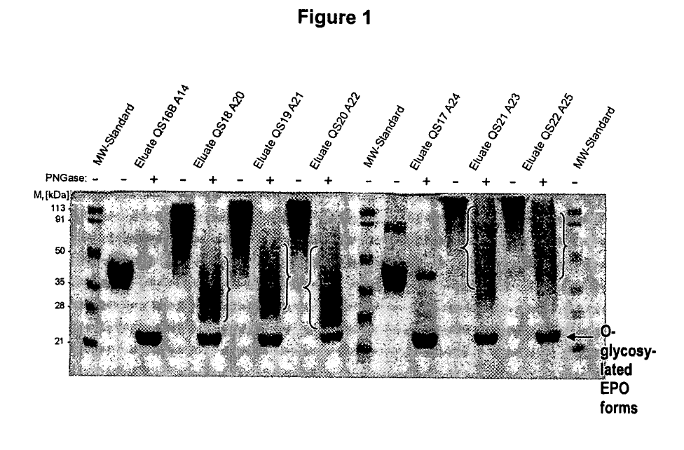

4 mg quantities of EPO, periodate oxidised EPO and HES-modified EPO forms were subjected to anion exchange chromatography on Q-Sepharose as described under Example 4. After buffer exchange to 50 mM Na-phosphate buffer pH 7.2 aliquots of samples were subjected to de-N-glycosylation by polypeptide N-glycosidase (PNGase) treatment (see Example 5.1). 5-10 μg aliquots of samples before and after PNGase treatment were subjected to SDS-PAGE analysis. As is depicted in FIG. 1, the samples A14 (=EPO) and A24 (periodate oxidised EPO incubated with unmodified HES) yielded the O-glycosylated EPO form after incubation with PNGase; whereas for samples A20, A21, A23 and A25 an additional diffuse Coomassie stained band was detected (indicated by brackets in FIG. 1) which represents EPO forms that are modified with HES at their O-glycan.

### Results 2

### RP-HPLC Separation of Liberated N-Glycans and de-N-glycosylated EPO Polypeptide

The de-N-glycosylated EPO forms were separated from liberated N-glycans by RP-HPLC as described in Example 5.2) and the resulting oligosaccharide fractions and the EPO protein were subjected to further analysis.

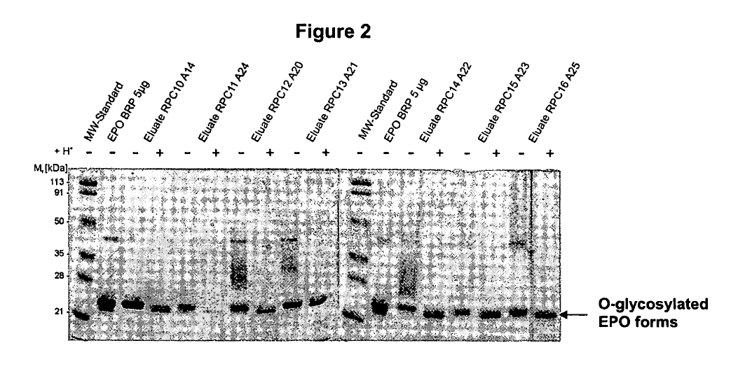

After SDS-PAGE analysis the EPO forms which were modified by HAS at their O-glycans (see FIG. 2) disappeared after mild acid treatment of the de-N-glycosylated samples which were previously isolated by RP-HPLC (see Example 5.2) indicating the acid labile nature of the modification (removal of sialic acid from the O-glycosylated EPO form by mild acid treatment is also indicated by the small shift of the non-hasylated protein band.

### Results 3

### Oligosaccharide Characterisation of Unmodified and HAS-Modified EPO Preparations

The oligosaccharide fractions obtained after RP-HPLC of PNGase-treated EPO forms were desalted and aliquots corresponding to 1-3 nmoles were subjected to HPAEC-PAD analysis and to compositional analysis as described under Example 5.5(c).

### Results 3(a)

### HPAEC-PAD Mapping of Native N-Glycans

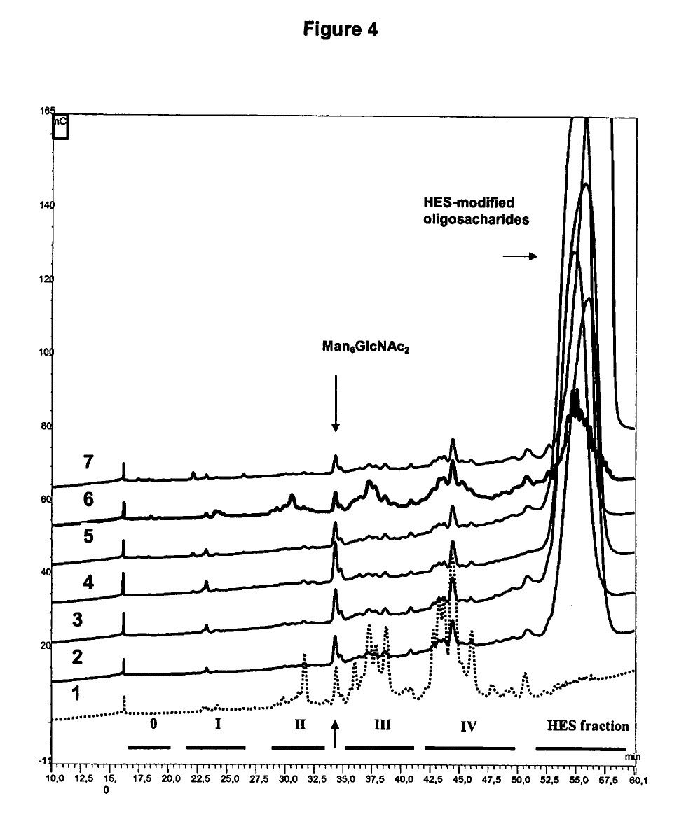

Mapping of the N-glycans of untreated EPO yielded an oligosaccharide pattern as depicted in FIG. 4. Based on peak response in HPAEC-PAD 0,7% of the oligosaccharide peak area was detected in the region of monosialylated glycans (22-25 min), 7% disialylated (28-32, 5 min), (4% Man₆-P=34,5 min), 30% trisialylated (36-41 min) and 58% tetrasialylated glycans (42-50 min).

After mild periodate oxidation of EPO the resulting N-glycans eluted as follows: 1,2% in the monosialo, 6.0 in the disialo, 2% in the Man6-P, 12% in the trisialo and 26% in the tetrasialo region whereas 50% of the HPAEC-PAD signal detected was observed at 51-57 min.

ESI-MS of the reduced and permethylated oligosaccharides indicated that about 80-85% of the N-acetylneuraminic acids were modified by periodate treatment (data not shown).

In the case of the oligosaccharide material obtained from HAS modified EPO preparations 90% of the HPAEC-PAD signal was detected at a retention time of 52-59 minutes (using a gradient as described in Example 5.4(b)) thus indicating that periodate oxidised EPO was HAS modified at almost completely all N-glycosylation sites.

### Results 3(b)

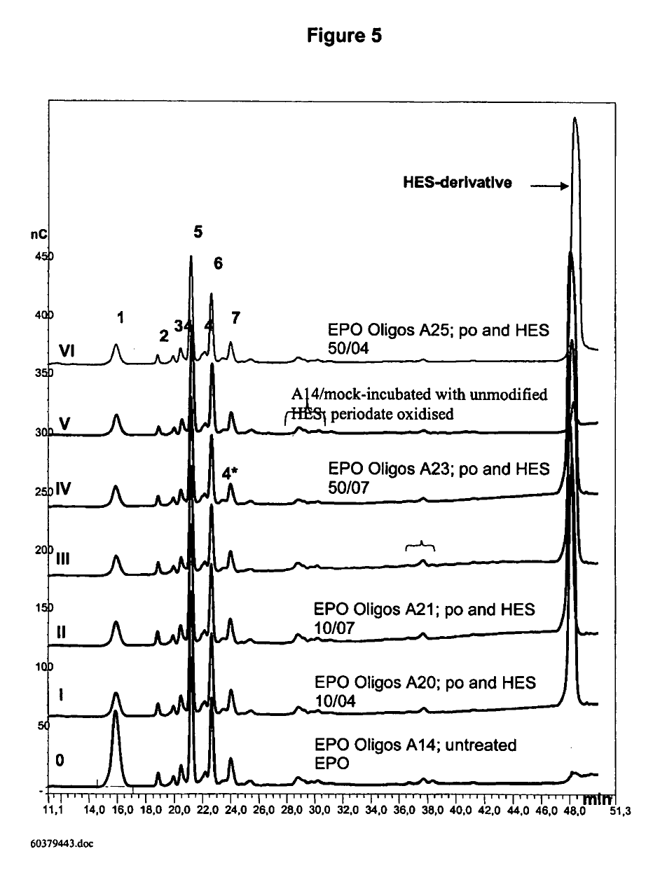

### HPAEC-PAD Mapping of N-glycans after Mild Acid Treatment (See FIG. 5)

Upon mild acid treatment (see Example 5.3) the oligosaccharides of EPO sample A14 yielded di-tri- and tetraantennary oligosaccharide signals which were identified by comparison of their retention time with standard reference complex-type structures. The %-peak areas for individual glycans structures are depicted in Table 6:

TABLE 6
Individual asialo oligosaccharide structures (% peak area) after mild acid
treatment observed in HPAEC-PAD analysis

| | | A20 | A21 | A23 | A24 | A25 |
|---|---|---|---|---|---|---|
| | A14 | (% | (% | (% | (% | (% |
| oligosaccharide- | (% peak | peak | peak | peak | peak | peak |
| structure | area) | area) | area)) | area) | area) | area) |
| diantennary | 2.8 | 3.0 | 3.0 | 2.9 | 2.1 | 2.6 |
| 2,4-triantennary | 2.8 | 3.0 | 2.9 | 3.0 | 2.4 | 2.8 |
| 2,6-triantennäay | 6.0 | 6.3 | 6.2 | 6.2 | 5.5 | 6.0 |
| Tetraantennary | 33.5 | 33.2 | 33.5 | 35.0 | 33.9 | 34.5 |
| Triantennary + 1R | 5.5 | 6.2 | 6.0 | 6.3 | 5.7 | 6.4 |
| Tetraantennary + 1R | 22.7 | 22.5 | 22.2 | 23.1 | 22.9 | 22.9 |
| Triantennary + 2R | 2.0 | 2.5 | 2.3 | 2.5 | 2.4 | 2.6 |
| Tetraantennary + 2R | 9.3 | 9.1 | 8.9 | 9.2 | 9.3 | 9.1 |
| Triantennary + 3R | 0.5 | 0.5 | 0.5 | 0.5 | 0.7 | 0.6 |
| Tetraantennary + 3R | 2.2 | 2.4 | 2.4 | 2.1 | 2.8 | 2.1 |
| Man₆-Phosphat | 2.8 | 2.4 | 2.8 | 2.2 | 1.9 | 1.8 |
| Residual | 10.2 | 9.1 | 9.3 | 6.9 | 10.4 | 8.6 |
| monosialylated | | | | | | |
| oligosaccharides* | | | | | | |

*due to the presence of O-acetylated N-acetylneuraminic acid residues which are partially resistent to mild acid treatment (see FIG. 5 indicated by a bracket, trace 2 from the top).

In summary, the starting material (A14), the mock HES incubated periodate oxidised EPO (A24) and the HES-modified EPO preparations exhibited an identical glycan pattern indicating that the derivatization procedures did not significantly affect the neutral carbohydrate structures. This was further confirmed by MALDI/TOF MS of the mild acid treated oligosaccharides revealing molecular ion signals at m=1809 (diantennary+proximal Fucose), m=2174 (triantennary+proximal Fucose), m=2539 (tetraantennary+proximal Fucose), m=2904 (tetraantennary+1 N-acetyllactosamin repeat+proximal Fucose), m=3269 (tetraantennary+2 N-acetyllactosamin repeat+proximal Fucose).

### Results 3(c)

### Compositional Analysis of Native N-Glycans

Native oligosaccharides of EPO isolated by RP-HPLC (see Example 5.2) were reduced and derivatised as described under Example 5.5(c). The data in Table 7 compares the monosaccharide composition of the oligosaccharides after de-N-glycosylation of EPO preparations yielding a fucose, mannose galactose and N-acetylglucosamine ratio of approximately 1:3:3.5:3 (values for GlcNAc-derivatives were low due to loss during the derivatisation procedure). The amount of the derivative for N-acetylneuraminic acid (NeuAc) detected in compositional analysis is in agreement with the amount of intact NeuAc observed in the desialylated N-glycan preparations in HPAEC-PA mapping.

TABLE 7
Compositional analyses of N-glycans from hasylated EPO-preparations
A14, A20-A25

| Sample: | Fuc | Man | Gal | Glc | GlcNAc | GlcHe1 | GlcHe2 | NeuNAc | Inositol |
|---|---|---|---|---|---|---|---|---|---|
| A14 | 1.17 | 3.00 | 3.23 | — | 2.33 | — | — | 0.74 | 6.83 |
| A21 | 1.10 | 3.00 | 3.61 | 15.81 | 2.79 | 6.51 | 1.03 | 0.90 | 9.85 |
| A23 | 1.03 | 3.00 | 3.85 | 73.78 | 2.16 | 32.08 | 5.15 | 0.92 | 13.41 |
| A24 | 0.68 | 3.00 | 3.40 | — | 2.55 | — | — | 0.62 | 9.08 |
| A20 | 1.04 | 3.00 | 3.13 | 43.5 | 2.99 | 2.74 | 1.07 | 1.11 | 3.27 |
| A21 | 1.01 | 3.00 | 2.40 | 15.05 | 1.98 | 3.68 | 1.47 | 0.84 | 2.01 |
| A25 | 1.18 | 3.00 | 2.86 | 148.9 | 1.90 | 40.99 | 1.99 | 0.78 | 2.02 |

Uncorrected results of the electronic integration of the peaks of the pertrimethylsilylated monosaccharide methylglycosides. 2.0 μg of Inositol was added to the samples as internal standard.

Information Sample code

| | |
|---|---|
| 0312-17/A14 = | EPO = starting material prior to periodate oxidation |
| 0401-09/A20 = | HES 10/04 periodate-oxidated |
| 0401-09/A21 = | HES 10/07 periodate-oxidated |
| 0401-09/A24 = | A14/mock-HES periodate-oxidated |
| 0401-09/A23 = | HES 50/07 periodate-oxidated |
| 0401-09/A25 = | HES 50/04 periodate-oxidated |

The detection of glucose and its mono- and di-hydroxethylated derivatives in the oligosaccharide preparations of A20, A21, A23 and A 25 confirmed the presence of HAS in this material and was not detected in the starting material (A14) or the material from EPO incubated with underivatised HES (A24).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of producing a conjugate of erythropoietin and hydroxyethyl starch, said method comprising reacting the hydroxyethyl starch with a crosslinking compound having two hydroxylamino groups to give a hydroxylamino functionalized hydroxyethyl starch derivative, and reacting the hydroxylamino group of said derivative with a carbohydrate moiety of the erythropoietin, wherein the hydroxyethyl starch has a mean molecular weight of at least 40 kD and a degree of substitution of at least 0.6.

2. The method as claimed in claim 1, wherein the hydroxyethyl starch has a mean molecular weight of at least 50 kD and a degree of substitution of at least 0.7.

3. The method as claimed in claim 1, wherein the hydroxyethyl starch has a mean molecular weight of about 50 kD and a degree of substitution of about 0.7 to 0.8.

4. The method as claimed in claim 1, wherein the crosslinking compound is O-[2-(2-aminooxy-ethoxy)-ethyl]-hydroxylamine.

5. The method as claimed in claim 1, wherein the hydroxyethyl starch is reacted with the crosslinking compound in an aqueous medium.

6. The method as claimed in claim 1, wherein the reaction is carried out at a pH of from 4.5 to 6.5.

7. The method as claimed in claim 1, wherein the reaction is carried out at a temperature of from 20 to 25° C.

8. The method as claimed in claim 1, wherein the hydroxylamino functionalized hydroxyethyl starch derivative is reacted with the carbohydrate moiety of the erythropoietin in an aqueous medium.

9. The method as claimed in claim 8, wherein the reaction is carried out at a pH of from 4.5 to 6.5.

10. The method as claimed in claim 8, wherein the reaction is carried out at a temperature of from 20 to 25° C.

11. The method as claimed in claim 1, wherein the carbohydrate moiety is an oxidized terminal saccharide unit of a carbohydrate side chain of the erythropoietin.

12. The method as claimed in claim 11, wherein the terminal saccharide unit is oxidized, optionally after partial or complete enzymatic or chemical or enzymatic and chemical removal of the terminal sialic acid.

13. The method as claimed in claim 11, wherein the terminal saccharide unit is galactose.

14. The method as claimed in claim 1, wherein the carbohydrate moiety is comprised in a carbohydrate side chain of the erythropoietin which was attached to the erythropoietin via N-linked or O-linked or N-linked and O-linked glycosylation during its production in mammalian cells, insect cells, or yeast cells.

15. A conjugate of erythropoietin and hydroxyethyl starch, obtainable by a method as claimed in claim 1.

16. A conjugate comprising hydroxyethyl starch, a crosslinking compound and erythropoietin, wherein the crosslinking compound is linked via an oxime linkage or an oxyamino group to the hydroxyethyl starch and via an oxime linkage to the carbohydrate moiety of the erythropoietin, and wherein the hydroxyethyl starch has a mean molecular weight of at least 40 kD and a degree of substitution of at least 0.6.

17. The conjugate as claimed in claim 16, wherein the hydroxyethyl starch has a mean molecular weight of at least 50 kD and a degree of substitution of at least 0.7.

18. The conjugate as claimed in claim 16, wherein the hydroxyethyl starch has a mean molecular weight of about 50 kD and a degree of substitution of about 0.7 to about 0.8.

19. The conjugate as claimed in claim 16, comprising a crosslinking compound having had two hydroxylamino groups, one of which being covalently linked to a carbohydrate moiety of the erythropoietin and one of which being covalently linked to the hydroxyethyl starch.

20. The conjugate as claimed in claim 16, wherein the crosslinking compound is O-[2-(2-aminooxy-ethoxy)-ethyl]-hydroxylamine.

21. The conjugate as claimed in claim 16, wherein the erythropoietin has the amino acid sequence of human erythropoietin.

22. The conjugate as claimed in claim 16, wherein the carbohydrate moiety is comprised in a carbohydrate side chain attached to the erythropoietin via N-linked or O-linked or N-linked and O-linked glycosylation, the erythropoietin comprising at least one carbohydrate side chain.

23. The conjugate as claimed in claim 22, wherein the at least one carbohydrate side chain was attached to the erythropoietin during the production of the erythropoietin in mammalian cells, insect cells, yeast cells, transgenic animals or transgenic plants.

24. The conjugate as claimed in claim 16, wherein the carbohydrate moiety is an oxidized galactose residue or a sialic acid residue.

25. A method for screening for a conjugate of erythropoietin and hydroxyalkyl starch, having improved in vivo activity compared to native erythropoietin, said method comprising the steps of (i) providing a candidate conjugate; (ii) testing the in vivo activity in comparison with native erythropoietin, wherein the mean molecular weight MW is varied in the range of from 1 to 300 kD and the degree of substitution DS is varied in the range of from 0.1 to 1.0, and wherein these parameters are simultaneously increased compared to a given combination of parameters.

26. The method as claimed in claim 25, wherein the given combination of parameters is a mean molecular weight MW of about 10 kD and a degree of substitution DS of about 0.4.

27. The method as claimed in claim 25, further comprising the step of incorporating the candidate conjugate into a therapeutic or prophylactic composition.

28. A method for the treatment of a human or animal body, comprising administering a conjugate as claimed in claim 15 to a human or animal in need of treatment.

29. A pharmaceutical composition comprising in a therapeutically effective amount a conjugate as claimed in claim 15.

30. A pharmaceutical composition as claimed in claim 29, further comprising at least one pharmaceutically acceptable diluent, adjuvant, or carrier.

31. A composition for the treatment of anemic disorders or hematopoietic dysfunction disorders or diseases related thereto, comprising a conjugate as claimed in claim 15.
